SEQUENCE LISTING (45 sequences)

SEQ ID NO: 1 - DNA, 16569 bp, Homo sapiens
  misc_feature (3107)..(3107): n is a, c, g, or t
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacnttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 2 - DNA, 783 bp, artificial
  cDNA
atggcccaccataattacccccatactccttacactattcctcatcacccaactaaaaatattaaacacaaactaccacctacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 3 - DNA, 300 bp, artificial
  cDNA
atgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaccctactcctaatcacataa

SEQ ID NO: 4 - DNA, 781 bp, Artificial
  cDNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 5 - DNA, 565 bp, Artificial
  cDNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 6 - DNA, 1174 bp, Artificial
  cDNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 7 - DNA, 1294 bp, Artificial
  cDNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 8 - DNA, 1228 bp, Artificial
  cDNA
atgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcct

SEQ ID NO: 9 - DNA, 522 bp, Artificial
  cDNA
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 10 - DNA, 582 bp, Artificial
  cDNA
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagccctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 11 - DNA, 2208 bp, Artificial
  cDNA
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 12 - DNA, 807 bp, Artificial
  cDNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 13 - DNA, 786 bp, Artificial
  cDNA
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 14 - DNA, 951 bp, Artificial
  cDNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 15 - DNA, 1905 bp, Artificial
  cDNA
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 16 - DNA, 1545 bp, Artificial
  cDNA
atgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 17 - DNA, 1629 bp, Artificial
  cDNA
ataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 18 - DNA, 642 bp, Artificial
  cDNA
atgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctccctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataa

SEQ ID NO: 19 - DNA, 129 bp, Artificial
  cDNA
atgccccaactaaatactaccgtatggcccaccataattacccccatactccttacactattcctcatcacccaactaaaaatattaaacacaaactaccacctacctccctcaccattggcagcctag

SEQ ID NO: 20 - DNA, 1147 bp, Artificial
  cDNA
atactactaatctcatcaatacaacccccgcccatcctacccagcacacacacaccgctgctaaccccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttat

SEQ ID NO: 21 - RNA, 783 nt, Homo sapiens
auggcccaccauaauuacccccauacuccuuacacuauuccucaucacccaacuaaaaauauuaaacacaaacuaccaccuaccucccucaccauuggcagccuagcauuagcaggaauaccuuuccucacagguuucuacuccaaagaccacaucaucgaaaccgcaaacauaucauacacaaacgccugagcccuaucuauuacucucaucgcuaccucccugacaagcgccuauagcacucgaauaauucuucucacccuaacaggucaaccucgcuuccccacccuuacuaacauuaacgaaaauaaccccacccuacuaaaccccauuaaacgccuggcagccggaagccuauucgcaggauuucucauuacuaacaacauuucccccgcaucccccuuccaaacaacaaucccccucuaccuaaaacucacagcccucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 22 - RNA, 300 nt, Homo sapiens
augccccucauuuacauaaauauuauacuagcauuuaccaucucacuucuaggaauacuaguauaucgcucacaccucauauccucccuacuaugccuagaaggaauaauacuaucgcuguucauuauagcuacucucauaacccucaacacccacucccucuuagccaauauugugccuauugccauacuagucuuugccgccugcgaagcagcggugggccuagcccuacuagucucaaucuccaacacauauggccuagacuacguacauaaccuaacccuacuccuaaucacauaa

SEQ ID NO: 23 - RNA, 781 nt, Homo sapiens
auggcacaugcagcgcaaguaggucuacaagacgcuacuuccccuaucauagaagagcuuaucaccuuucaugaucacgcccucauaaucauuuuccuuaucugcuuccuaguccuguaugcccuuuuccuaacacucacaacaaaacuaacuaauacuaacaucucagacgcucaggaaauagaaaccgucugaacuauccugcccgccaucauccuaguccucaucgcccucccaucccuacgcauccuuuacauaacagacgaggucaacgaucccucccuuaccaucaaaucaauuggccaccaaugguacugaaccuacgaguacaccgacuacggcggacuaaucuucaacuccuacauacuucccccauuauuccuagaaccaggcgacccagacaauuauacccuagccaaccccuuaaacaccccuccccacaucaagcccgaaugauauuuccuauucgccuacacaauucuccgauccgucccuaacaaacuaggaggcguccuugcccuauuacuauccauccucauccuagcaauaauccccauccuccauauauccaaacaacaaagcauaauauuucgcccacuaagccaaucacuuuauugacuccuagccgcagaccuccucauucuaaccugaaucggaggacaaccaguaagcuacccuuuuaccaucauuggacaaguagcauccguacuauacuucacaacaauccuaauccuaauaccaacuaucucccuaauugaaaacaaaauacucaaaugggccu

SEQ ID NO: 24 - RNA, 565 nt, Homo sapiens
auggcacaugcagcgcaaguaggucuacaagacgcuacuuccccuaucauagaagagcuuaucaccuuucaugaucacgcccucauaaucauuuuccuuaucugcuuccuaguccuguaugcccuuuuccuaacacucacaacaaaacuaacuaauacuaacaucucagacgcucaggaaauagaaaccgucugaacuauccugcccgccaucauccuaguccucaucgcccucccaucccuacgcauccuuuacauaacagacgaggucaacgaucccucccuuaccaucaaaucaauuggccaccaaugguacugaaccuacgaguacaccgacuacggcggacuaaucuucaacuccuacauacuucccccauuauuccuagaaccaggcgaccugcgacuccuagccgcagaccuccucauucuaaccugaaucggaggacaaccaguaagcuacccuuuuaccaucauuggacaaguagcauccguacuauacuucacaacaauccuaauccuaauaccaacuaucucccuaauugaaaacaaaauacucaaaugggccu

SEQ ID NO: 25 - RNA, 1174 nt, Homo sapiens
auggcacaugcagcgcaaguaggucuacaagacgcuacuuccccuaucauagaagagcuuaucaccuuucaugaucacgcccucauaaucauuuuccuuaucugcuuccuaguccuguaugcccuuuuccuaacacucacaacaaaacuaacuaauacuaacaucucagacgcucaggaaauagaaaccgucugaacuauccugcccgccaucauccuaguccucaucgcccucccaucccuacgcauccuuuacauaacagacgaggucaacgaucccucccuuaccaucaaaucaauuggccaccaaugguacugaaccuacgaguacaccgacuacggcggacuaaucuucaacuccuacauacuucccccauuauuccuagaaccaggcgaccugcgacuccuugacguugacaaucgaguaguacucccgauugaagcccccauucguauaauaauuacaucacaagacgucuugcacucaugagcuguccccacauuaggcuuaaaaacagaugcaauucccggacgucuaaaccaaaccacuuucaccgcuacacgaccggggguauacuacggucaaugcucugaaaucuguggagcaaaccacaguuucaugcccauauucuugcacgaaacgggaucaaacaacccccuaggaaucaccucccauuccgauaaaaucaccuuccacccuuacuacacaaucaaagacgcccucggcuuacuucucuuccuucucuccuuaaugacauuaacacuauucucaccagaccuccuaggcgacccagacaauuauacccuagccaaccccuuaaacaccccuccccacaucaagcccgaaugauauuuccuauucgccuacacaauucuccgauccgucccuaacaaacuaggaggcguccuugcccuauuacuauccauccucauccuagcaauaauccccauccuccauauauccaaacaacaaagcauaauauuucgcccacuaagccaaucacuuuauugacuccuagccgcagaccuccucauucuaaccugaaucggaggacaaccaguaagcuacccuuuuaccaucauuggacaaguagcauccguacuauacuucacaacaauccuaauccuaauaccaacuaucucccuaauugaaaacaaaauacucaaaugggccu

SEQ ID NO: 26 - RNA, 1294 nt, Homo sapiens
augaacgaaaaucuguucgcuucauucauugcccccacaauccuaggccuacccgccgcaguacugaucauucuauuucccccucuauugauccccaccuccaaauaucucaucaacaaccgacuaaucaccacccaacaaugacuaaucaaacuaaccucaaaacaaaugauaaccauacacaacacuaaaggacgaaccugaucucuuauacuaguauccuuaaucauuuuuauugccacaacuaaccuccucggacuccugccucacucauuuacaccaaccacccaacuaucuauaaaccuagccaugcacuacucaccagacgccucaaccgccuuuucaucaaucgcccacaucacucgagacguaaauuauggcugaaucauccgcuaccuucacgccaauggcgccucaauauucuuuaucugccucuuccuacacaucgggcgaggccuauauuacggaucauuucucuacucagaaaccugaaacaucggcauuauccuccugcuugcaacuauagcaacagccuucauaggcuauguccucccgugaggccaaauaucauucugaggggccacaguaauuacaaacuuacuauccgccaucccauacauugggacagaccuaguucaaugaaucugaggaggcuacucaguagacagucccacccucacacgauucuuuaccuuucacuucaucuugcccuucauuauugcagcccuagcaacacuccaccuccuauucuugcacgaaacgggaucaaacaacccccuaggaaucaccucccauuccgauaaaaucaccuuccacccuuacuacacaaucaaagacgcccucggcuuacuucucuuccuucucuccuuaaugacauuaacacuauucucaccagaccuccuaggcgacccagacaauuauacccuagccaaccccuuaaacaccccuccccacaucaagcccgaaugauauuuccuauucgccuacacaauucuccgauccgucccuaacaaacuaggaggcguccuugcccuauuacuauccauccucauccuagcaauaauccccauccuccauauauccaaacaacaaagcauaauauuucgcccacuaagccaaucacuuuauugacuccuagccgcagaccuccucauucuaaccugaaucggaggacaaccaguaagcuacccuuuuaccaucauuggacaaguagcauccguacuauacuucacaacaauccuaauccuaauaccaacuaucucccuaauugaaaacaaaauacucaaaugggccu

SEQ ID NO: 27 - RNA, 1228 nt, Homo sapiens
augccccucauuuacauaaauauuauacuagcauuuaccaucucacuucuaggaauacuaguauaucgcucacaccucauauccucccuacuaugccuagaaggaauaauacuaucgcuguucauuauagcuacucucauaacccucaacacccacucccucuuagccaauauugugccuauugccauacuagucuuuggcgccugccugauccuccaaaucaccacaggacuauuccuagccaugcacuacucaccagacgccucaaccgccuuuucaucaaucgcccacaucacucgagacguaaauuauggcugaaucauccgcuaccuucacgccaauggcgccucaauauucuuuaucugccucuuccuacacaucgggcgaggccuauauuacggaucauuucucuacucagaaaccugaaacaucggcauuauccuccugcuugcaacuauagcaacagccuucauaggcuauguccucccgugaggccaaauaucauucugaggggccacaguaauuacaaacuuacuauccgccaucccauacauugggacagaccuaguucaaugaaucugaggaggcuacucaguagacagucccacccucacacgauucuuuaccuuucacuucaucuugcccuucauuauugcagcccuagcaacacuccaccuccuauucuugcacgaaacgggaucaaacaacccccuaggaaucaccucccauuccgauaaaaucaccuuccacccuuacuacacaaucaaagacgcccucggcuuacuucucuuccuucucuccuuaaugacauuaacacuauucucaccagaccuccuaggcgacccagacaauuauacccuagccaaccccuuaaacaccccuccccacaucaagcccgaaugauauuuccuauucgccuacacaauucuccgauccgucccuaacaaacuaggaggcguccuugcccuauuacuauccauccucauccuagcaauaauccccauccuccauauauccaaacaacaaagcauaauauuucgcccacuaagccaaucacuuuauugacuccuagccgcagaccuccucauucuaaccugaaucggaggacaaccaguaagcuacccuuuuaccaucauuggacaaguagcauccguacuauacuucacaacaauccuaauccuaauaccaacuaucucccuaauugaaaacaaaauacucaaaugggccu

SEQ ID NO: 28 - RNA, 522 nt, Homo sapiens
auguucgccgaccguugacuauucucuacaaaccacaaagacauuggaacacuauaccuauuauucggcgcaugagcuggaguccuaggcacagcucuaagccuccuuauucgagccgagcugggccagccaggcaaccuucuagguaacgaccacaucuacaacguuaucgucacagcccucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 29 - RNA, 582 nt, Homo sapiens
auguucgccgaccguugacuauucucuacaaaccacaaagacauuggaacacuauaccuauuauucggcgcaugagcuggaguccuaggcacagcucuaagccuccuuauucgagccgagcugggccagccaggcaaccuucuagguaacgaccacaucuacaacguuaucgucacagcccaugcauuuguaauaaucuucuucauaguaauacccaucauaaucggaggcuuuggcaacugacuaguuccccuaauaaucggugcccccgauauggcguuuccccgcauaaacaacauaagcuucugacucuuaccucccucucuccuacuccugcucgcaucugcuauaguggaggccggagcaggaacagguugaacagucuacccucccuuagcagggaacuacucccacccuggagcccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 30 - RNA, 2208 nt, Homo sapiens
auguucgccgaccguugacuauucucuacaaaccacaaagacauuggaacacuauaccuauuauucggcgcaugagcuggaguccuaggcacagcucuaagccuccuuauucgagccgagcugggccagccaggcaaccuucuagguaacgaccacaucuacaacguuaucgucacagcccaugcauuuguaauaaucuucuucauaguaauacccaucauaaucggaggcuuuggcaacugacuaguuccccuaauaaucggugcccccgauauggcguuuccccgcauaaacaacauaagcuucugacucuuaccucccucucuccuacuccugcucgcaucugcuauaguggaggccggagcaggaacagguugaacagucuacccucccuuagcagggaacuacucccacccuggagccuccguagaccuaaccaucuucuccuuacaccuagcaggugucuccucuaucuuaggggccaucaauuucaucacaacaauuaucaauauaaaacccccugccauaacccaauaccaaacgccccucuucgucugauccguccuaaucacagcaguccuacuucuccuaucucucccaguccuagcugcuggcaucacuauacuacuaacagaccgcaaccucaacaccaccuucuucgaccccgccggaggaggagaccccauucuauaccaacaccuauucugauuuuucggucacccugaaguuuauauucuuauccuaccaggcuucggaauaaucucccauauuguaacuuacuacuccggaaaaaaagaaccauuuggauacauagguauggucugagcuaugauaucaauuggcuuccuaggguuuaucgugugagcacaccauauauuuacaguaggaauagacguagacacacgagcauauuucaccuccgcuaccauaaucaucgcuauccccaccggcgucaaaguauuuagcugacucgccacacuccacggaagcaauaugaaaugaucugcugcagugcucugagcccuaggauucaucuuucuuuucaccguagguggccugacuggcauuguauuagcaaacucaucacuagacaucguacuacacgacacguacuacguuguagcccacuuccacuauguccuaucaauaggagcuguauuugccaucauaggaggcuucauucacugauuuccccuauucucaggcuacacccuagaccaaaccuacgccaaaauccauuucacuaucauauucaucggcguaaaucuaacuuucuucccacaacacuuucucggccuauccggaaugccccgacguuacucggacuaccccgaugcauacaccacaugaaacauccuaucaucuguaggcucauucauuucucuaacagcaguaauauuaauaauuuucaugauuugagaagccuucgcuucgaagcgaaaaguccuaauaguagaagaacccuccauaaaccuggagugacuauauggaugccccccacccuaccacacauucgaagaacccguauacauaaaagcaggaauaccuuuccucacagguuucuacuccaaagaccacaucaucgaaaccgcaaacauaucauacacaaacgccugagcccuaucuauuacucucaucgcuaccucccugacaagcgccuauagcacucgaauaauucuucucacccuaacaggucaaccucgcuuccccacccuuacuaacauuaacgaaaauaaccccacccuacuaaaccccauuaaacgccuggcagccggaagccuauucgcaggauuucucauuacuaacaacauuucccccgcaucccccuuccaaacaacaaucccccucuaccuaaaacucacagcccucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 31 - RNA, 807 nt, Homo sapiens
auggcacaugcagcgcaaguaggucuacaagacgcuacuuccccuaucauagaagagcuuaucaccuuucaugaucacgcccucauaaucauuuuccuuaucugcuuccuaguccuguaugcccuuuuccuaacacucacaacaaaacuaacuaauacuaacaucucagacgcucaggaaauagaaaccgcaaacauaucauacacaaacgccugagcccuaucuauuacucucaucgcuaccucccugacaagcgccuauagcacucgaauaauucuucucacccuaacaggucaaccucgcuuccccacccuuacuaacauuaacgaaaauaaccccacccuacuaaaccccauuaaacgccuggcagccggaagccuauucgcaggauuucucauuacuaacaacauuucccccgcaucccccuuccaaacaacaaucccccucuaccuaaaacucacagcccucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 32 - RNA, 786 nt, Homo sapiens
auggcacaugcagcgcaaguaggucuacaagacgcuacuuccccuaucauagaagagcuuaucaccuuucaugaucacgcccucauaaucauuuuccuuaucugcuuccuaguccuguaugcccuuuuccuaacacucacaacaaaacuaacuaauacuaacaucucagacgcucaggaaauagaaaccgucugaacuauccugcccgccaucauccuaguccucaucgcccucccaucccuacgcauccuuuacauaacagacgaggucaacgaucccucccuuaccaucaaaucaauuggccaccaaugguacugaaccuacgaguacaccgacuacggcggacuaaucuucaacuccuacauacuucccccauuauuccuagaaccaggcgaccugcgacuccuugacguugacaaucgaguaguacucccgauugaagcccccauucguauaauaauuacaucacaagacgucuugcacucaugagcuguccccacauuaggcuuaaaaacagaugcaauucccggacgucuaaaccaaaccacuuucaccgcuacacgaccggggguauacuacggucaaugcucugaaaucuguggagcaaaccacaguuucaugcccaucguccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 33 - RNA, 951 nt, Homo sapiens
augaacgaaaaucuguucgcuucauucauugcccccacaauccuaggccuacccgccgcaguacugaucauucuauuucccccucuauugauccccaccuccaaauaucucaucaacaaccgacuaaucaccacccaacaaugacuaaucaaacuaaccucaaaacaaaugauaaccauacacaacacuaaaggacgaaccugaucucuuauacuaguauccuuaaucauuuuuauugccacaacuaaccuccucggacuccugccucacucauuuacaccaaccacccaacuaucuauaaaccuagccauggccauccccuuaugagcgggcacagugauuauaggcuuucgcucuaagauuaaaaaugcccuagcccacuucuuaccacaaggcacaccuacaccccuuauccccauacuaguuauuaucgaaaccaucagccuacucauucaaccaauagcccuggccguacgccuaaccgcuaacauuacugcaggccaccuacucaugcaccuaauuggaagcgccacccuagcaauaucaaccauuaaccuucccucuacacuuaucaucuucacaauucuaauucuacugacuauccuagaaaucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 34 - RNA, 1905 nt, Homo sapiens
augaacgaaaaucuguucgcuucauucauugcccccacaauccuaggccuacccgccgcaguacugaucauucuauuucccccucuauugauccccaccuccaaauaucucaucaacaaccgacuaaucaccacccaacaaugacuaaucaaacuaaccucaaaacaaaugauaaccauacacaacacuaaaggacgaaccugaucucuuauacuaguauccuuaaucauuuuuauugccacaacuaaccuccucggacuccugccucacucauuuacaccaaccacccaacuaucuauaaaccuagccauggccauccccuuaugagcgggcacagugauuauaggcuuucgcucuaagauuaaaaaugcccuagcccacuucuuaccacaaggcacaccuacaccccuuauccccauacuaguuauuaucgaaaccaucagccuacucauucaaccaauagcccuggccguacgccuaaccgcuaacauuacugcaggccaccuacucaugcaccuaauuggaagcgccacccuagcaauaucaaccauuaaccuucccucuacacuuaucaucuucacaauucuaauucuacugacuauccuagaaaucgcugucgccuuaauccaagccuacguuuucacacuucuaguaagccucuaccuacacuccaacucaugagacccacaacaaauagcccuucuaaacgcuaauccaagccucaccccacuacuaggccuccuccuagcagcagcaggcaaaucagcccaauuaggucuccaccccugacuccccucagccauagaaggccccaccccagucucagcccuacuccacucaagcacuauaguuguagcaggaaucuucuuacucauccgcuuccacccccuagcagaaaauagcccacuaauccaaacucuaacacuaugcuuaggcgcuaucaccacucuguucgcagcagucugcgcccuuacacaaaaugacaucaaaaaaaucguagccuucuccacuucaagucaacuaggacucauaauaguuacaaucggcaucaaccaaccacaccuagcauuccugcacaucuguacccacgccuucuucaaagccauacuauuuaugugcuccggguccaucauccacaaccuuaacaaugaacaagauauucgaaaaauaggaggacuacucaaaaccauaccucucacuucaaccucccucaccauuggcagccuagcauuagcaggaauaccuuuccucacagguuucuacuccaaagaccacaucaucgaaaccgcaaacauaucauacacaaacgccugagcccuaucuauuacucucaucgcuaccucccugacaagcgccuauagcacucgaauaauucuucucacccuaacaggucaaccucgcuuccccacccuuacuaacauuaacgaaaauaaccccacccuacuaaaccccauuaaacgccuggcagccggaagccuauucgcaggauuucucauuacuaacaacauuucccccgcaucccccuuccaaacaacaaucccccucuaccuaaaacucacagcccucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 35 - RNA, 1545 nt, Homo sapiens
augacccaccaaucacaugccuaucauauaguaaaacccagcccaugaccccuaacaggggcccucucagcccuccuaaugaccuccggccuagccaugugauuucacuuccacuccauaacgcuccucauacuaggccuacuaaccaacacacuaaccauauaccaaugauggcgcgauguaacacgagaaagcacauaccaaggccaccacacaccaccuguccaaaaaggccuucgauacgggauaauccuauuuauuaccucagaaguuuuuuucuucgcaggauuuuucugagccuuuuaccacuccagccuagccccuaccccccaauuaggagggcacuggcccccaacaggcaucaccccacuacuaggccuccuccuagcagcagcaggcaaaucagcccaauuaggucuccaccccugacuccccucagccauagaaggccccaccccagucucagcccuacuccacucaagcacuauaguuguagcaggaaucuucuuacucauccgcuuccacccccuagcagaaaauagcccacuaauccaaacucuaacacuaugcuuaggcgcuaucaccacucuguucgcagcagucugcgcccuuacacaaaaugacaucaaaaaaaucguagccuucuccacuucaagucaacuaggacucauaauaguuacaaucggcaucaaccaaccacaccuagcauuccugcacaucuguacccacgccuucuucaaagccauacuauuuaugugcuccggguccaucauccacaaccuuaacaaugaacaagauauucgaaaaauaggaggacuacucaaaaccauaccucucacuucaaccucccucaccauuggcagccuagcauuagcaggaauaccuuuccucacagguuucuacuccaaagaccacaucaucgaaaccgcaaacauaucauacacaaacgccugagcccuaucuauuacucucaucgcuaccucccugacaagcgccuauagcacucgaauaauucuucucacccuaacaggucaaccucgcuuccccacccuuacuaacauuaacgaaaauaaccccacccuacuaaaccccauuaaacgccuggcagccggaagccuauucgcaggauuucucauuacuaacaacauuucccccgcaucccccuuccaaacaacaaucccccucuaccuaaaacucacagcccucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 36 - RNA, 1629 nt, Homo sapiens
auaaacuucgccuuaauuuuaauaaucaacacccuccuagccuuacuacuaauaauuauuacauuuugacuaccacaacucaacggcuacauagaaaaauccaccccuuacgagugcggcuucgacccuauaucccccgcccgcgucccuuucuccauaaaauucuucuuaguagcuauuaccuucuuauuauuugaucuagaaauugcccuccuuuuaccccuaccaugagcccuacaaacaacuaaccugccacuaauaguuaugucaucccucuuauuaaucaucauccuagcccuaagucuggccaacacagcagccauucaagcaauccuauacaaccguaucggcgauaucgguuucauccucgccuuagcaugauuuauccuacacuccaacucaugagacccacaacaaauagcccuucuaaacgcuaauccaagccucaccccacuacuaggccuccuccuagcagcagcaggcaaaucagcccaauuaggucuccaccccugacuccccucagccauagaaggccccaccccagucucagcccuacuccacucaagcacuauaguuguagcaggaaucuucuuacucauccgcuuccacccccuagcagaaaauagcccacuaauccaaacucuaacacuaugcuuaggcgcuaucaccacucuguucgcagcagucugcgcccuuacacaaaaugacaucaaaaaaaucguagccuucuccacuucaagucaacuaggacucauaauaguuacaaucggcaucaaccaaccacaccuagcauuccugcacaucuguacccacgccuucuucaaagccauacuauuuaugugcuccggguccaucauccacaaccuuaacaaugaacaagauauucgaaaaauaggaggacuacucaaaaccauaccucucacuucaaccucccucaccauuggcagccuagcauuagcaggaauaccuuuccucacagguuucuacuccaaagaccacaucaucgaaaccgcaaacauaucauacacaaacgccugagcccuaucuauuacucucaucgcuaccucccugacaagcgccuauagcacucgaauaauucuucucacccuaacaggucaaccucgcuuccccacccuuacuaacauuaacgaaaauaaccccacccuacuaaaccccauuaaacgccuggcagccggaagccuauucgcaggauuucucauuacuaacaacauuucccccgcaucccccuuccaaacaacaaucccccucuaccuaaaacucacagcccucgcugucacuuuccuaggacuucuaacagcccuagaccucaacuaccuaaccaacaaacuuaaaauaaaauccccacuaugcacauuuuauuucuccaacauacucggauucuacccuagcaucacacaccgcacaauccccuaucuaggccuucuuacgagccaaaaccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 37 - RNA, 642 nt, Homo sapiens
augcuaaaacuaaucgucccaacaauuauauuacuaccacugacaugacuuuccaaaaaacacauaauuugaaucaacacaaccacccacagccuaauuauuagcaucaucccucuacuauuuuuuaaccaaaucaacaacaaccuauuuagcuguuccccaaccuuuuccuccgacccccuaacaaccccccuccuaauacuaacuaccugacuccuaccccucacaaucauggcaagccaacgccacuuauccagugaaccacuaucacgaaaaaaacucuaccucucuauacuaaucucccuacaaaucuccuuaauuauaacauucacagccacagaacuaaucauauuuuauaucuucuucgaaaccacacuuauccccaccuuggcuaucaucacccgaugaggcaaccagccagaacgccugaacgcaggcacauacuuccuauucuacacccuaguaggcucccugccccuacuccuccuagaccuaaccugacuagaaaagcuauuaccuaaaacaauuucacagcaccaaaucuccaccuccaucaucaccucaacccaaaaaggcauaauuaaacuuuacuuccucucuuucuucuucccacucauccuaacccuacuccuaaucacauaa

SEQ ID NO: 38 - RNA, 129 nt, Homo sapiens
augccccaacuaaauacuaccguauggcccaccauaauuacccccauacuccuuacacuauuccucaucacccaacuaaaaauauuaaacacaaacuaccaccuaccucccucaccauuggcagccuag

SEQ ID NO: 39 - RNA, 1147 nt, Homo sapiens
auacuacuaaucucaucaauacaacccccgcccauccuacccagcacacacacaccgcugcuaaccccauaccccgaaaauguugguuauacccuucccguacuaauuaauccccuggcccaacccgucaucuacucuaccaucuuugcaggcacacucaucacagcgcuaagcucgcacugauuuuuuaccugaguaggccuagaaauaaacaugcuagcuuuuauuccaguucuaaccaaaaaaauaaacccucguuccacagaagcugccaucaaguauuuccucacgcaagcaaccgcauccauaauccuucuaauagcuauccucuucaacaauauacucuccggacaaugaaccauaaccaauacuaccaaucaauacucaucauuaauaaucauaauagcuauagcaauaaaacuaggaauagcccccuuucacuucugagucccagagguuacccaaggcaccccucugacauccggccugcuucuucucacaugacaaaaacuagcccccaucucaaucauauaccaaaucucucccucacuaaacguaagccuucuccucacucucucaaucuuauccaucauagcaggcaguugagguggauuaaaccaaacccagcuacgcaaaaucuuagcauacuccucaauuacccacauaggaugaauaauagcaguucuaccguacaacccuaacauaaccauucuuaauuuaacuauuuauauuauccuaacuacuaccgcauuccuacuacucaacuuaaacuccagcaccacgacccuacuacuaucucgcaccugaaacaagcuaacaugacuaacacccuuaauuccauccacccuccucucccuaggaggccugcccccgcuaaccggcuuuuugcccaaaugggccauuaucgaagaauucacaaaaaacaauagccucaucauccccaccaucauagccaccaucacccuccuuaaccucuacuucuaccuacgccuaaucuacuccaccucaaucacacuacuccccauaucuaacaacguaaaaauaaaaugacaguuugaacauacaaaacccaccccauuccuccccacacucaucgcccuuaccacgcuacuccuaccuaucuccccuuuuauacuaauaaucuuau

SEQ ID NO: 40 - protein, 100 aa, Artificial
  putative protein sequence
  misc_feature (100)..(100): Xaa can be any naturally occurring amino acid
MPLIYMNIMLAFTISLLGMLVYRSHLMSSLLCLEGMMLSLFIMATLMTLNTHSLLANIVPIAMLVFAACEAAVGLALLVSISNTYGLDYVHNLTLLLITX

SEQ ID NO: 41 - protein, 261 aa, Artificial
  putative protein sequence
  misc_feature (261)..(261): Xaa can be any naturally occurring amino acid
MAHAAQVGLQDATSPIMEELITFHDHALMIIFLICFLVLYALFLTLTTKLTNTNISDAQEMETVWTILPAIILVLIALPSLRILYMTDEVNDPSLTIKSIGHQWYWTYEYTDYGGLIFNSYMLPPLFLEPGDPDNYTLANPLNTPPHIKPEWYFLFAYTILRSVPNKLGGVLALLLSILILAMIPILHMSKQQSMMFRPLSQSLYWLLAADLLILTWIGGQPVSYPFTIIGQVASVLYFTTILILMPTISLIENKMLKWAX

SEQ ID NO: 42 - protein, 269 aa, Artificial
  putative protein sequence
  misc_feature (269)..(269): Xaa can be any naturally occurring amino acid
MAHAAQVGLQDATSPIMEELITFHDHALMIIFLICFLVLYALFLTLTTKLTNTNISDAQEMETANMSYTNAWALSITLIATSLTSAYSTRMILLTLTGQPRFPTLTNINENNPTLLNPIKRLAAGSLFAGFLITNNISPASPFQTTIPLYLKLTALAVTFLGLLTALDLNYLTNKLKMKSPLCTFYFSNMLGFYPSITHRTIPYLGLLTSQNLPLLLLDLTWLEKLLPKTISQHQISTSIITSTQKGMIKLYFLSFFFPLILTLLLITX

SEQ ID NO: 43 - protein, 262 aa, Artificial
  putative protein sequence
  misc_feature (262)..(262): Xaa can be any naturally occurring amino acid
MAHAAQVGLQDATSPIMEELITFHDHALMIIFLICFLVLYALFLTLTTKLTNTNISDAQEMETVWTILPAIILVLIALPSLRILYMTDEVNDPSLTIKSIGHQWYWTYEYTDYGGLIFNSYMLPPLFLEPGDLRLLDVDNRVVLPIEAPIRMMITSQDVLHSWAVPTLGLKTDAIPGRLNQTTFTATRPGVYYGQCSEICGANHSFMPIVLDLTWLEKLLPKTISQHQISTSIITSTQKGMIKLYFLSFFFPLILTLLLITX

SEQ ID NO: 44 - protein, 43 aa, Artificial
  putative protein sequence
  misc_feature (43)..(43): Xaa can be any naturally occurring amino acid
MPQLNTTVWPTMITPMLLTLFLITQLKMLNTNYHLPPSPLAAX

SEQ ID NO: 45 - protein, 383 aa, Artificial
  putative protein sequence
  misc_feature (383)..(383): Xaa can be any naturally occurring amino acid (or part thereof)
MLLISSMQPPPILPSTHTPLLTPYPENVGYTLPVLINPLAQPVIYSTIFAGTLITALSSHWFFTWVGLEMNMLAFIPVLTKKMNPRSTEAAIKYFLTQATASMILLMAILFNNMLSGQWTMTNTTNQYSSLMIMMAMAMKLGMAPFHFWVPEVTQGTPLTSGLLLLTWQKLAPISIMYQISPSLNVSLLLTLSILSIMAGSWGGLNQTQLRKILAYSSITHMGWMMAVLPYNPNMTILNLTIYIILTTTAFLLLNLNSSTTTLLLSRTWNKLTWLTPLIPSTLLSLGGLPPLTGFLPKWAIIEEFTKNNSLIIPTIMATITLLNLYFYLRLIYSTSITLLPMSNNVKMKWQFEHTKPTPFLPTLIALTTLLLPISPFMLMILX

(19) United States
(12) Patent Application Publication
(10) Pub. No.: US 2018/0346985 A1
(43) Pub. Date: Dec. 6, 2018

(54) UV Associated mtDNA Fusion Transcripts and Methods and Uses Thereof

(71) Applicant: MDNA Life Sciences Inc., Wilmington, DE (US)

(72) Inventors: Andrew Harbottle, Tyne and Wear (GB); Gabriel Dakubo, Thunder Bay (CA); Ryan L. Parr, Thunder Bay (CA); Jennifer Creed, Broomsfield, CO (US); Brian Reguly, Thunder Bay (CA); Kerry Robinson, Thunder Bay (CA)

(21) Appl. No.: 16/057,052

(22) Filed: Aug. 7, 2018

Related U.S. Application Data
(63) Continuation of application No. 15/347,601, filed on Nov. 9, 2016, now abandoned.
Application No. 15/347,601 is a division of application No. 14/569,147, filed on Dec. 12, 2014, now abandoned.
Application No. 14/569,147 is a continuation of application No. 13/582,049, filed on Apr. 2, 2013, now abandoned.
(60) Provisional application No. 61/309,216, filed on Mar. 1, 2010.

(51) Int. Cl. C12Q 1/6883; C12N 9/12; C12N 9/02; C07K 14/80; C12N 15/62; C12N 9/14

(52) U.S. Cl. CPC C12Q 1/6883; C12Y 306/01003; C12Y 207/10001; C12Y 106/99003; C12Y 109/03001; C12N 9/12; C12N 9/0036; C12N 9/14; C12N 9/0053; C07K 2319/00; C07K 14/80; C12N 15/62; C12Q 2600/148; C12Q 2600/156

(57) ABSTRACT

The present invention provides novel mitochondrial fusion transcripts and related deletion molecules that are associated with UV exposure. Methods for in vivo and in vitro detection of mtDNA molecules and associated fusion transcripts is also provided, as is their use in the screening and testing of skin care products.

### CROSS REFERENCE TO RELATED APPLICATIONS

This application is a Continuation of application Ser. No. 15/347,601, filed Nov. 9, 2016, which is a Division of U.S. patent application Ser. No. 14/569,147, filed Dec. 12, 2014, which is a Continuation of U.S. patent application Ser. No. 13/582,049, filed Apr. 2, 2013, which is a national entry of PCT Patent Application Number PCT/CA2011/050120, filed Mar. 1, 2011, which claims the benefit of priority under 35 U.S.C. § 119(e) of U.S. Provisional Patent Application No. 61/309,216, filed Mar. 1, 2010. Each of the aforementioned applications is incorporated by reference herein as if set forth in its entirety.

### STATEMENT REGARDING SEQUENCE LISTING

The Sequence Listing associated with this application is provided in text format submitted electronically via EFS-Web, and is hereby incorporated by reference into the specification.

### FIELD OF THE INVENTION

The present invention relates to the field of mitochondrial genomics. In particular, the invention relates to mitochondrial fusion transcripts and related deletion molecules associated with UV exposure, as well as their detection and monitoring in biological samples.

### BACKGROUND OF THE INVENTION

Mitochondrial Genome

The mitochondrial genome is a compact yet critical sequence of nucleic acids. Mitochondrial DNA, or “mtDNA”, comprises a small genome of 16,569 nucleic acid base pairs (bp) (Anderson et al., 1981; Andrews et al., 1999) in contrast to the immense nuclear genome of 3.3 billion bp (haploid). The mtDNA genetic complement is substantially smaller than that of its nuclear cell mate (0.0005%). However, individual cells carry anywhere from 10³ to 10⁴ mitochondria depending on specific cellular functions (Singh and Modica-Napolitano, 2002). Communication or chemical signalling routinely occurs between the nuclear and mitochondrial genomes (Sherratt et al., 1997). Moreover, specific nuclear components are responsible for the maintenance and integrity of mitochondrial sequences (Croteau et al., 1999). All mtDNA genomes in a given individual are identical due to the clonal expansion of mitochondria within the ovum, once fertilization has occurred. However mutagenic events can induce sequence diversity reflected as somatic mutations. These mutations may accumulate in different tissues throughout the body in a condition known as heteroplasmy.

Mitochondrial Fusion Transcriptome

The mitochondrial genome is unusual in that it is a circular, intron-less DNA molecule. The genome is interspersed with repeat motifs which flank specific lengths of sequences. Sequences between these repeats are prone to deletion under circumstances which are not well understood. Given the number of repeats in the mitochondrial genome, there are many possible deletions. The best known example is the 4977 “common deletion.” This deletion has been associated with several purported conditions and diseases and is thought to increase in frequency with aging (Dai et al., 2004; Ro et al., 2003; Barron et al., 2001; Lewis et al., 2000; Muller-Hocker, 1998; Porteous et al., 1998).

Various other mitochondrial deletions have been associated with early onset of prostate, skin and lung cancer, as well as aging (e.g. Polyak et al., 1998), premature aging, and exposure to carcinogens (Lee et al., 1998). Additionally, researchers have found that ultraviolet radiation (UV) is important in the development and pathogenesis of non-melanoma skin cancer (NMSC) (Weinstock 1998; Rees, 1998) and that UV induces mtDNA damage in human skin (Birch-Machin, 2000a). In Canadian Patent Application No. 2,480,184, for example, a 3895 bp deletion in the minor arc of the mitochondrial genome was identified as a biomarker of UV-induced DNA damage. The 3895 bp deletion has also since been associated with skin cancer (PCT Application No. PCT/CA2006/000652).

As discussed in the Applicant's co-pending International Patent Application No. PCT/CA2009/000351, the knowledge gained from mapping large-scale deletions of the human mitochondrial genome has been useful in the identification of deletions associated with disease. Computer analysis of the mitochondrial genome, for example, has allowed the Applicant to identify deletion sites that, upon initiation of a deletion event in the DNA molecule, re-close or re-ligate to produce a fused DNA sequence having an open reading frame (ORF). From these studies, the Applicant identified a subset of deletion molecules and associated fusion transcripts that showed relevance to malignancy.

The Applicant has identified a further subset of mitochondrial deletions and fusion transcripts that are associated with UV exposure. Results from these investigations and their application in detecting UV damage are described herein.

### SUMMARY OF THE INVENTION

In one aspect, the present invention provides a method of detecting mitochondrial fusion transcripts.

In another aspect, the present invention provides a method of detecting a mitochondrial fusion transcript, wherein the transcript is associated with UV exposure, comprising the steps of: (a) providing a biological sample; and (b) detecting the presence of the mitochondrial fusion transcript in the sample.

In accordance with another aspect of the invention, there is provided a method for determining the cumulative UV exposure in a subject, comprising the steps of: (a) providing a biological sample from the subject; and (b) detecting the presence of a mitochondrial fusion transcript associated with UV exposure.

In accordance with another aspect of the invention, there is provided an isolated mitochondrial fusion transcript associated with UV exposure.

In accordance with another aspect of the invention, there is provided use of a fusion transcript for detecting UV exposure.

In accordance with another aspect of the invention, there is provided use of a fusion transcript as a biomarker for UV exposure.

In accordance with another aspect of the invention, there is provided a kit for detecting UV exposure, the kit comprising: (a) a probe having a sequence substantially complementary to a portion of an isolated fusion sequence of the invention.

In accordance with another aspect of the invention, there is provided a method of detecting a deletion in the human mtDNA genome, wherein said deletion is associated with UV exposure, the method comprising the steps of: (a) providing a biological sample; (b) extracting mtDNA from the biological sample; and (c) detecting the presence of a mtDNA deletion molecule.

In accordance with another aspect of the invention, there is provided a method for determining the cumulative UV exposure in a subject, comprising the steps of: (a) providing a biological sample; (b) extracting mtDNA from the biological sample; and (c) detecting the presence of a deletion in the mtDNA associate with UV exposure.

In accordance with another aspect of the invention, there is provided an isolated mtDNA deletion molecule associated with UV exposure.

In accordance with another aspect of the invention, there is provided use of a mtDNA deletion for detecting UV exposure.

In accordance with another aspect of the invention, there is provided use of a mtDNA deletion as a biomarker for UV exposure.

In accordance with another aspect of the invention, there is provided a method of testing the efficacy of a skin care product to prevent, minimize, ameliorate or protect against UV exposure or damage, the method comprising the steps of: (a) preparing a skin care product with the desired characteristics; (b) applying the skin care product to a patient's skin or a skin equivalent; (c) exposing the skin or skin equivalent to UVR; (d) detecting the presence of a mitochondrial fusion transcript associated with UV exposure in a sample of the exposed patient's skin or the exposed skin equivalent; and (e) comparing the presence of the transcript to a reference value.

In accordance with another aspect of the invention, there is provided a method of testing the efficacy of a new skin care product or formulation to prevent, minimize, ameliorate or protect against UV exposure or damage, the method comprising the steps of: (a) preparing a skin care product with the desired characteristics; (b) applying the skin care product to a patient's skin or a skin equivalent; (c) exposing the skin or skin equivalent to UVR; (d) detecting the presence of a mtDNA deletion molecule in a sample of the exposed patient's skin or the exposed skin equivalent, wherein the mtDNA deletion molecule is associated with UV exposure; and (e) comparing the presence of the molecule to a reference value.

In accordance with another aspect of the invention, there is provided a method of screening skin care products for the ability to prevent, minimize, ameliorate or protect against UV exposure or damage, the method comprising the steps of: (a) applying the skin care products to a patient's skin or a skin equivalent; (b) exposing the skin or skin equivalent to UVR; (c) detecting the presence of a mitochondrial fusion transcript associated with UV exposure; and (e) comparing the presence of the fusion transcript for each skin care product tested against a reference value and/or each other.

In accordance with another aspect of the invention, there is provided a method of screening skin care products for the ability to prevent, minimize, ameliorate or protect against UV exposure or damage, the method comprising the steps of: (a) applying the skin care product to a patient's skin or a skin equivalent; (b) exposing the skin or skin equivalent to UVR; (c) detecting the presence of a mtDNA deletion molecule in a sample of the exposed patient's skin or the exposed skin equivalent, wherein the mtDNA deletion molecule is associated with UV exposure; and (e) comparing the presence of the deletion molecule for each skin care product tested against a reference value and/or each other.

In accordance with another aspect of the invention, there is provided a method of testing the efficacy of a skin care product to prevent, ameliorate or protect against skin aging or photo-aging, the method comprising the steps of: (a) preparing a skin care product with the desired characteristics; (b) applying the skin care product to a patient's skin or a skin equivalent; (c) exposing the skin or skin equivalent to UVR; (d) detecting the presence of a mitochondrial fusion transcript associated with UV exposure in a sample of the exposed patient's skin or the exposed skin equivalent; and (e) comparing the presence of the transcript to a reference value.

In accordance with another aspect of the invention, there is provided a method of testing the efficacy of a new skin care product or formulation to prevent, ameliorate or protect against skin aging or photo-aging, the method comprising the steps of: (a) preparing a skin care product with the desired characteristics; (b) applying the skin care product to a patient's skin or a skin equivalent; (c) exposing the skin or skin equivalent to UVR; (d) detecting the presence of a mtDNA deletion molecule in a sample of the exposed patient's skin or the exposed skin equivalent, wherein the mtDNA deletion molecule is associated with UV exposure; and (e) comparing the presence of the molecule to a reference value.

In accordance with another aspect of the invention, there is provided a method of screening skin care products for the ability to prevent, ameliorate or protect against skin aging or photo-aging, the method comprising the steps of: (a) applying the skin care products to a patient's skin or a skin equivalent; (b) exposing the skin or skin equivalent to UVR; (c) detecting the presence of a mitochondrial fusion transcript associated with UV exposure; and (e) comparing the presence of the fusion transcript for each skin care product tested against a reference value and/or each other.

In accordance with another aspect of the invention, there is provided a method of screening skin care products for the ability to prevent, ameliorate or protect against skin aging or photo-aging, the method comprising the steps of: (a) applying the skin care product to a patient's skin or a skin equivalent; (b) exposing the skin or skin equivalent to UVR; (c) detecting the presence of a mtDNA deletion molecule in a sample of the exposed patient's skin or the exposed skin equivalent, wherein the mtDNA deletion molecule is associated with UV exposure; and (e) comparing the presence of the deletion molecule for each skin care product tested against a reference value and/or each other.

### BRIEF DESCRIPTION OF THE DRAWINGS

These and other features of the invention will become more apparent in the following detailed description in which reference is made to the appended drawings.

FIG. 1 shows an amino acid sequence of Frame 2 from positions 386-547:4443-5511 after removal of nucleotide positions 548-4442.

FIG. 2 shows the reading frames produced after the removal of nucleotide positions 548-4442. Nucleotide 382 is denoted by a red box identifying the beginning of Reading Frame 2 in relation to position 1 of the mitochondrial genome.

FIG. 3 shows the contributing sequences of the Frame 2 fusion transcript formed by the removal of nucleotide positions 548-4442.

FIG. 4 shows the final amino acid sequence of the fusion transcript form by the deletion of nucleotide positions 548-4442. The sequence includes the recombined nucleotide positions 470-547 and 4443-5511 of the mitochondrial genome.

FIG. 5A shows an in vivo UVR dose response using mtDNA deletion analysis.

FIG. 5B shows a sample calculation for determining UV damage.

FIGS. 6-9 show the expression of fusion transcripts 2, 3, 11, 12, 20 and 32 of the present invention following UV dosing in skin equivalents.

FIGS. 10-12 show in vivo and in vitro testing of three UV formulations.

FIGS. 13 and 14 show screening of various sunscreen and anti-aging brands following UV exposure.

FIG. 15 shows the UV spectrum used for the solar-simulated irradiation of Example 3.

FIG. 16 shows the UV spectrum used for the solar-simulated irradiation of Example 4.

### DETAILED DESCRIPTION OF THE INVENTION

The present invention provides novel mitochondrial fusion transcripts and related mtDNA molecules that are associated with UV exposure. The invention also provides for the detection and monitoring of fusion transcripts and associated mtDNA molecules in biological samples. As well, the invention provides for the use of fusion transcripts and related mtDNA deletions in the screening and testing of skin care products.

The techniques and procedures of the invention are generally performed according to conventional methods in the art and various general references (see, for example, Sambrook et al. Molecular Cloning: A Laboratory Manual, 2d ed. (1989) Cold Spring Harbor Laboratory Press, Cold Spring Harbor, N.Y., and Lakowicz, J. R. Principles of Fluorescence Spectroscopy, New York: Plenum Press (1983)).

### Definitions

Unless defined otherwise, all technical and scientific terms used herein have the same meaning as commonly understood by one of ordinary skill in the art to which this invention belongs. The following terms, unless otherwise indicated, shall be understood to have the following meanings:

As used herein, the term “about” is intended to refer to a variation from the stated value or factor. It is to be understood that such a variation is always included in any given value or factor provided herein, whether or not it is specifically referred to. By way of example, such variation may be approximately +1-10%. It is also well known to all persons skilled in the art that there are degrees of error necessarily associated with measurement. The degree of error will vary depending on the precision of the instrument used to take the reading. Since the Applicant cannot know the precision of the instrument that will be used by persons working the invention, the degree of measurement error cannot be necessarily defined. Likewise, the accuracy of the nucleotide and amino acid sequences submitted herewith is dependent on the accuracy of the equipment and process used. Therefore, minor sequence variations are not considered to fall outside of the teaching of this application.

As used herein, the expression “mitochondrial fusion transcript” or “fusion transcript” refers to an RNA transcription product produced as a result of the transcription of a mutated mitochondrial DNA sequence wherein such mutations may comprise mitochondrial deletions and other large-scale mitochondrial DNA rearrangements.

The term “hybridize,” as used herein, refers to the ability of a nucleic acid to bind detectably and specifically to a second nucleic acid. Polynucleotides, oligonucleotides and fragments thereof hybridize to direct target nucleic acid strands under hybridization and wash conditions that minimize appreciable amounts of detectable binding to non-specific nucleic acids. High stringency conditions can be used to achieve selective hybridization conditions as known in the art. Typically, hybridization and washing conditions are performed at high stringency according to conventional hybridization procedures. Washing conditions are generally 1-3×SSC, 0.1-1% SDS, 50-70° C. with a change of wash solution after about 5-30 minutes.

The term “ameliorate” includes the arrest, prevention, decrease, or improvement in one or more the symptoms, signs, or features of UV damage, both temporary and long-term.

As used herein, “aberration” or “mutation” encompasses modifications in the wild type mitochondrial DNA sequence that results in a fusion transcript and includes, without limitation, substantive or large-scale mtDNA deletions.

As used herein, “mitochondrial DNA” or “mtDNA” is DNA present in or originated in mitochondria.

The term “subject” or “patient” as used herein refers to an animal in need of treatment or an animal being tested.

The term “animal,” as used herein, refers to both human and non-human animals, including, but not limited to, mammals, birds and fish.

As used herein the term “derived from,” as applied to an object indicates that the object is obtained from a specified source, albeit not necessarily directly from that source.

As used herein, “diagnostic” or “diagnosing” means using the presence or absence or quantity of a mutation or combination of mutations as a factor in UV exposure diagnosis or UV exposure management.

The term “skin” refers to the outer protective covering of the body, consisting of the corium and the epidermis, and is understood to include sweat and sebaceous glands, as well as hair follicle structures. In one embodiment, the skin is mammalian skin, preferably human.

As used herein, “biological sample” refers to a tissue or bodily fluid containing cells from which a molecule of interest can be obtained. For example, the biological sample can be derived from skin cells or tissue, wherein tissue may be taken from the dermis or epidermis, or a combination of both. The biological sample can be used either directly as obtained from the source or following a pre-treatment to modify the character of the sample. The sample may be obtained by a variety of methods including, but not limited to, punch biopsy, surgical excision, and non-invasive or minimally invasive skin sampling methods such as a wet swabbing, tapelift, cotton tip swabbing, scraping of skin using a sterile surgical blade, scraping of skin using a wooden scraper, sticky surface of an adhesive pad (CapSure™ Clean-up Pad, Arcturus), film from LCM MacroCap™ (Arcturus), heated film from LCM MacroCap™ (Arcturus) and employing a small gauge needle (for example, 28 gauge), to collect micro-cores of skin tissue. These methods are well known in the art (see, for example, Applicant's co-pending applications PCT/CA2007/001790 and PCT/CA2008/001801, which disclose various methods for collecting skin samples).

Genomic Mutations

MtDNAs are useful biomarkers in the identification of risk factors or disruptive cellular processes associated with disease onset and environmental exposure to factors such as toxins, carcinogens or harmful radiation. According to the present invention, large-scale rearrangement mutations in the mitochondrial genome result in the identification of fusion transcripts associated with UV exposure. Thus, the use of mtDNA encoding such transcripts and probes directed thereto for the detection, diagnosis and monitoring of UV exposure is provided herein.

The methodologies of the present invention are also useful in the identification of skin care products either through the detection of mtDNA deletions or their associated fusion transcripts. Thus, the present application provides for various methods to detect (and measure) the amount of UV related damage in a biological sample, as well as to screen and test for products that can reduce or prevent such damage.

One of skill in the art will appreciate that the mtDNA molecules for use in the methods of the present invention may be derived through the isolation of naturally-occurring mutants or may be based on the complementary sequence of any of the fusion transcripts described herein. Exemplary mtDNA sequences and fusion transcripts are disclosed in the Applicant's co-pending PCT application (PCT/CA2009/000351), which is incorporated herein by reference.

Detection of Mutant Genomic Sequences

Mutant sequences associated with UV exposure according to the present invention comprise mtDNA deletions that result in the generation of a fusion transcript. While the modification or change can vary greatly in size from only a few bases to several kilobases, preferably the modification results in a substantive or large-scale mtDNA deletion, also termed genomic aberration.

Techniques for extracting and isolating aberrant mtDNA molecules have been previously disclosed in the Applicant's co-pending patent application (PCT/CA2009/000351). Such methods, which also include techniques for selecting appropriate primers, probes and genomic sequences (i.e. those having a novel mtDNA junction point), are incorporated herein by reference.

According to an aspect of the present invention, to determine candidate genomic sequences, a junction point of a sequence deletion is first identified. Sequence deletions are primarily identified by direct and indirect repetitive elements which flank the sequence to be deleted at the 5′ and 3′ end. The removal of a section of the nucleotides from the genome followed by the ligation of the genome results in a fused DNA sequence with an open reading frame (ORF) and novel junction point.

Exemplary mtDNA molecules for use in the methods of the present invention are provided below. As previously described (see PCT/CA2009/000351), these mtDNAs are based on modifications of the known mitochondrial genome (SEQ ID NO: 1) and have been assigned a fusion or “FUS” designation, wherein A:B represents the junction point between the last mitochondrial nucleotide of the first spliced gene and the first mitochondrial nucleotide of the second spliced gene. The identification of the spliced genes is provided in parentheses followed
by the corresponding sequence identifier. Where provided below, (AltMet) and (OrigMet) refer to alternate and original translation start sites, respectively.
FUS 8469:13447 (AltMet) (ATP synthase F0 subunit 8 (ATPase8) to NADH dehydrogenase subunit 5 (ND5)) (SEQ ID No: 2)
FUS 10744:14124 (NADH dehydrogenase subunit 4L (ND4L) to NADH dehydrogenase subunit 5 (ND5)) (SEQ ID No: 3)
FUS 7974:15496 (Cytochrome c oxidase subunit II (COII) to Cytochrome b (Cytb)) (SEQ ID No: 4)
FUS 7992:15730 (Cytochrome c oxidase subunit II (COM) to Cytochrome b (Cytb)) (SEQ ID No: 5)
FUS 8210:15339 (Cytochrome c oxidase subunit II (COM) to Cytochrome b (Cytb)) (SEQ ID No: 6)
FUS 8828:14896 (ATP synthase FO subunit 6 (ATPase6) to Cytochrome b (Cytb)) (SEQ ID No: 7)
FUS 10665:14856 (NADH dehydrogenase subunit 4L (ND4L) to Cytochrome b (Cytb)) (SEQ ID No: 8)
FUS 6075:13799 (Cytochrome c oxidase subunit I (COI) to NADH de hydrogenase subunit 5 (ND5)) (SEQ ID No: 9)
FUS 6325:13989 (Cytochrome c oxidase subunit I (COI) to NADH dehydrogenase subunit 5 (ND5)) (SEQ ID No: 10)
FUS 7438:13476 (Cytochrome c oxidase subunit I (COI) to NADH dehydrogenase subunit 5 (ND5)) (SEQ ID No: 11)
FUS 7775:13532 (Cytochrome c oxidase subunit II (COM) to NADH dehydrogenase subunit 5 (ND5)) (SEQ ID No: 12)
FUS 8213:13991 (Cytochrome c oxidase subunit II (COM) to NADH dehydrogenase subunit 5 (ND5)) (SEQ ID No: 13)
FUS 9144:13816 ((ATP synthase FO subunit 6 (ATPase6) to NADH dehydrogenase subunit 5 (ND5)) (SEQ ID No: 14)
FUS 9191:12909 (ATP synthase FO subunit 6 (ATPase6) to NADH dehydrogenase subunit 5 (ND5)) (SEQ ID No: 15)
FUS 9574:12972 (Cytochrome c oxidase subunit III (COIII) to NADH dehydrogenase subunit 5 (ND5)) (SEQ ID No: 16)
FUS 10367:12829 (NADH dehydrogenase subunit 3 (ND3) to NADH dehydrogenase subunit 5 (ND5)) (SEQ ID No: 17)
FUS 11232:13980 (NADH dehydrogenase subunit 4 (ND4) to NADH dehydrogenase subunit 5 (ND5)) (SEQ ID No: 18)
FUS 8469:13447 (OrigMet) (ATP synthase FO subunit 8 (ATPase 8) to NADH dehydrogenase subunit 5 (ND5)) (SEQ ID No: 19)
FUS 547:4443 (Met to NADH dehydrogenase subunit 2 (ND2)) (SEQ ID No: 20)

As described in the examples below, once the sequences are identified their association with UV exposure is analysed. Such testing may be carried out in vivo in a subject or patient (see Example 5), or through the testing of skin cultures that are grown and dosed with varying levels of UVR (see Examples 3 and 4). Expression of these sequences may also be monitored in respect of one or more house keeper genes including, but not limited to, Human PPIB, Human PGK-1, Human ACTB (beta actin), Human GUSB (Beta Glucuronidase), Human B2M (Beta-2-microglobulin), Human PPIA (peptidylprolyl isomerase A (cyclophilin A)), Human GAPD (glyeraldehyde-3-phosphate dehydrogenase), Human HPRT1 (hypoxanthine phosporibosyltransferase 1), Human RPLPO=LRP (ribosmal protein, large, PO), Human HMBS=PBGD (hydroxymethlbilane synthase), Human TBP (TATA box binding protein), Human TRFC (transferrin receptor (p90, CD71), and Human ABCC13 (Chromosome 13—psuedogene).

In one embodiment of the invention, the following mtDNA sequences are determined useful for predicting, diagnosing or monitoring UV damage, and for testing and screening skin care products effective in preventing or ameliorating UV damage:
SEQ ID NO: 3 (FUS 10744:14124)
SEQ ID NO: 4 (FUS 7974:15496)
SEQ ID NO: 12 (FUS 7775:13532)
SEQ ID NO: 13 (FUS 8213:13991)
SEQ ID NO: 19 (FUS 8469:13447; OrigMet)
SEQ ID NO: 20 (FUS 547:4443).

In another embodiment of the invention, mtDNA sequences having SEQ ID NOs: 19 and 20 are preferably used in carrying out the methods of the invention.

In addition to the above sequences, the present invention also provides the use of variants or fragments of these sequences for predicting, diagnosing and/or monitoring UV damage or exposure.

“Variant”, as used herein, refers to a nucleic acid differing from a mtDNA sequence of the present invention, but retaining essential properties thereof. Generally, variants are overall closely similar, and, in many regions, identical to a select mtDNA sequence. Specifically, the variants of the present invention comprise at least one of the nucleotides of the junction point of the spliced genes, and may further comprise one or more nucleotides adjacent thereto. In one embodiment of the invention, the variant sequence is at least 80%, 85%, 90%, 95%, 96%, 97%, 98% or 99% identical to any one of the mtDNA sequences of the invention, or the complementary strand thereto.

In the present invention, “fragment” refers to a short nucleic acid sequence which is a portion of that contained in the disclosed genomic sequences, or the complementary strand thereto. This portion includes at least one of the nucleotides comprising the junction point of the spliced genes, and may further comprise one or more nucleotides adjacent thereto. The fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases of any one of the mtDNA sequences listed above. In this context “about” includes the particularly recited value, a value larger or smaller by several (5, 4, 3, 2, or 1) nucleotides, at either terminus or at both termini. These fragments have uses that include, but are not limited to, as diagnostic probes and primers as discussed herein. Of course, larger fragments (e.g., 50, 150, 500, 600, 2000 nucleotides) are also contemplated.

Primers and Probes

Another aspect of the invention is to provide a primer or probe capable of recognizing an mtDNA sequence of the invention. As used herein, the terms “primer” and “probe” refer to an oligonucleotide which forms a duplex structure with a sequence in the target nucleic acid, due to complementarity of at least one sequence in the primer or probe with a sequence in the target region. The probe may be labeled, according to methods known in the art.

Once aberrant mtDNA associated with UV exposure is identified, hybridization of mtDNA to, for example, an array of oligonucleotides can be used to identify particular mutations, however, any known method of hybridization may be used.

The preparation of suitable primers and probes for use in the present invention may be generated as described in the Applicant's co-pending PCT application. In this regard, probes can be generated directly against exemplary mtDNA fusion molecules of the invention, or to a fragment or variant thereof. For instance, the sequences set forth in SEQ ID NOs: 3, 4 12, 13, 19 and 20 can be used to design primers or probes that will detect a nucleic acid sequence comprising a fusion sequence of interest. As would be understood by those of skill in the art, primers or probes which hybridize to these nucleic acid molecules may do so under highly stringent hybridization conditions or lower stringency conditions, such conditions known to those skilled in the art and found, for example, in Current Protocols in Molecular Biology (John Wiley & Sons, New York (1989)), 6.3.1-6.3.6. Primers and probes of the invention may also hybridize to target sequences where there is DNA slippage or where a similar sequence altering event has occurred.

In specific embodiments of the invention, the probes of the invention contain a sequence complementary to at least a portion of the aberrant mtDNA comprising the junction point of the spliced genes. This portion includes at least one of the nucleotides involved in the junction point A:B, and may further comprise one or more nucleotides adjacent thereto. In this regard, the present invention encompasses any suitable targeting mechanism that will select an mtDNA molecule using the nucleotides involved and/or adjacent to the junction point A:B.

Various types of probes (as described in the Applicant's co-pending PCT application) are contemplated by the present invention. The probes of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A probe of “at least 20 nt in length,” for example, is intended to include 20 or more contiguous bases that are complementary to an mtDNA sequence of the invention. Of course, larger probes (e.g., 50, 150, 500, 600, 2000 nucleotides) may be preferable.

The probes of the invention will also hybridize to nucleic acid molecules in biological samples, thereby enabling the methods of the invention. Accordingly, in one aspect of the invention, there is provided a hybridization probe for use in detecting, diagnosing and/or monitoring UV damage or exposure, wherein the probe is complementary to at least a portion of an aberrant mtDNA molecule. In another aspect the present invention provides probes and a use of (or a method of using) such probes for screening or testing skin care products effective in preventing or ameliorating UV damage or exposure.

Assays

Measuring the level of aberrant mtDNA in a biological sample can determine the level of UV exposure in a subject. The present invention, therefore, encompasses methods for detecting, diagnosing or monitoring UV damage or exposure, comprising obtaining one or more biological samples, extracting mtDNA from the samples, and assaying the samples for aberrant mtDNA by: quantifying the amount of one or more aberrant mtDNA sequences in the sample and comparing the quantity detected with a reference value.

As would be understood by those of skill in the art, the reference value is based on whether the method seeks to detect, diagnose or monitor UV damage or exposure. Accordingly, the reference value may relate to mtDNA data collected from one or more known UV exposed biological samples, from one or more known non-UV exposed biological samples, and/or from one or more biological samples taken over time. The sample may be derived from rarely sun exposed, occasionally sun exposed, usually sun exposed skin or blood and collected by a variety of methods including, but not limited to, punch biopsy, surgical excision, and non-invasive or minimally invasive skin sampling methods such as a wet swabbing, tapelift, cotton tip swabbing, scraping of skin using a sterile surgical blade, scraping of skin using a wooden scraper, sticky surface of an adhesive pad (CapSure™ Clean-up Pad, Arcturus), film from LCM MacroCap™ (Arcturus), heated film from LCM MacroCap™ (Arcturus) and employing a small gauge needle (for example, 28 gauge), to collect micro-cores of skin tissue.

The step of detecting the presence of mutations in the mtDNA can be selected from any technique as is known to those skilled in the art. For example, analyzing mtDNA can comprise sequencing the mtDNA, amplifying mtDNA by PCR, Southern, Northern, Western South-Western blot hybridizations, denaturing HPLC, hybridization to microarrays, biochips or gene chips, molecular marker analysis, biosensors, melting temperature profiling or a combination of any of the above.

In one aspect, the invention provides a method of detecting UV exposure in a mammal, the method comprising assaying a tissue sample from the mammal for the presence of an aberrant mitochondrial DNA described above. The present invention also provides for methods comprising assaying a tissue sample from the mammal by hybridizing the sample with at least one hybridization probe. The probe may be generated against a mutant mitochondrial DNA sequence of the invention as described herein.

In another aspect, the invention provides a method as above, wherein the assay comprises: a) conducting a hybridization reaction using at least one of the probes of the invention to allow the at least one probe to hybridize to a complementary aberrant mitochondrial DNA sequence; b) quantifying the amount of the at least one aberrant mitochondrial DNA sequence in the sample by quantifying the amount of the mitochondrial DNA hybridized to the at least one probe; and, c) comparing the amount of the mitochondrial DNA in the sample to at least one known reference value.

Also included in the present invention are methods for detecting, diagnosing or monitoring UV damage or exposure comprising diagnostic imaging assays as known in the art. The diagnostic assays of the invention can be readily adapted for high-throughput. High-throughput assays provide the advantage of processing many samples simultaneously and significantly decrease the time required to screen a large number of samples. The present invention, therefore, contemplates the use of the nucleotides of the present invention in high-throughput screening or assays to detect and/or quantitate target nucleotide sequences in a plurality of test samples.

Fusion Transcripts

Another aspect of the invention is the identification of fusion transcripts and associated hybridization probes useful in methods for predicting, diagnosing and/or monitoring UV damage or exposure. As disclosed in the Applicant's co-pending PCT application, such molecules may be derived through the isolation of naturally-occurring transcripts or, alternatively, by the recombinant expression of mtDNAs isolated according to the methods of the invention. These mtDNAs typically comprise a spliced gene having the initiation codon from the first gene and the termination codon of the second gene. Accordingly, fusion transcripts derived therefrom include a junction point associated with the spliced genes.

Detection of Fusion Transcripts

Naturally occurring fusion transcripts can be extracted from a biological sample and identified according to any suitable method known in the art, or may be conducted according to the methods described in the Applicant's co-pending PCT application. In one embodiment of the invention, stable polyadenylated fusion transcripts are identified using Oligo(dT) primers that target transcripts with poly-A tails, followed by RT-PCR using primer pairs designed against the target transcript.

The following exemplary fusion transcripts were detected using these methods:
SEQ ID NO: 22 (Transcript 2; 10744:14124)
SEQ ID NO: 38 (Transcript 20; 8469:13447; OrigMet)

Fusion transcripts can also be produced by recombinant techniques known in the art. Typically this involves transformation (including transfection, transduction, or infection) of a suitable host cell with an expression vector comprising an mtDNA sequence of interest.

Detection of Unexpected Fusion Transcripts

Fusion transcripts of an unexpected nature have also been identified by the Applicant and proven useful in the methods of the present invention. Until now, each of the fusion transcripts identified occurred as a result of a deletion event in the mitochondrial genome resulting in the fusion of two genes to form a novel sequence then transcribed by the mitochondria. By contrast, the 3895 bp deletion described in detail below, results in the fusion of the D-loop, which is located in a non-coding region of the mtDNA.

Why Detection of 3895 bp Deletion Associated Fusion Transcript is Unexpected

The majority of mitochondrial fusion transcripts are formed by the deletion of nucleotide content between two adjacent or non-adjacent genes (see Genomic Mutations section above and the Applicant's co-pending PCT application). After removal of the deleted DNA, the remainder of the mitochondrial genome is then recombined resulting in a new gene, or transcript. As previously discussed, this spliced gene is comprised of the initiation codon of the 5′-most original gene, varying contributions of genetic content from the original two genes, and the termination codon of the 3′-most original gene.

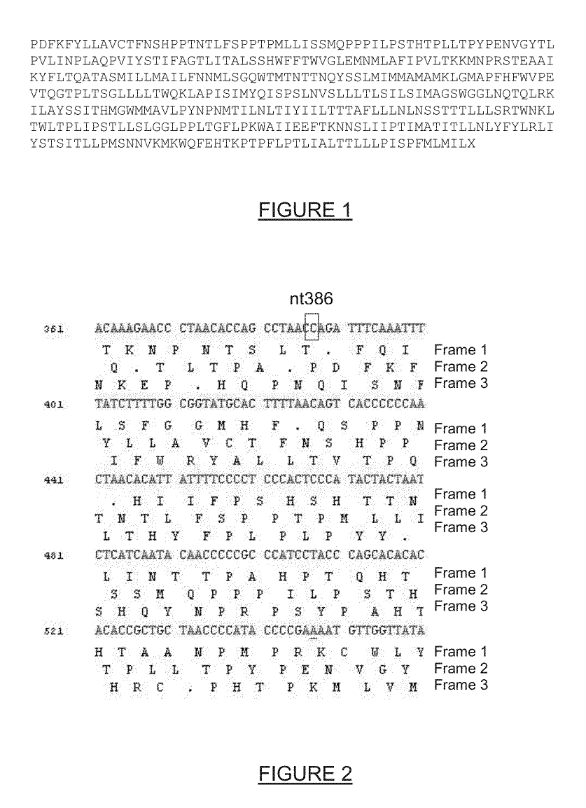

By contrast, the fusion transcript from the deletion of the genetic content between nucleotide positions 547-4443 (Transcript 32; SEQ ID NO: 39) is unexpected, since the 5′ portion is located within the non-coding Displacement Loop (D-Loop) (16024-576) of the mitochondrial genome. Despite this fact, when positions 548-4442 are removed an intact reading frame in Frame 2 is observed from positions 386-547:4443-5511 (see FIGS. 1 & 2). Frames 1 and 3 have many termination codons, thus failing to produce a reading frame (FIG. 2).

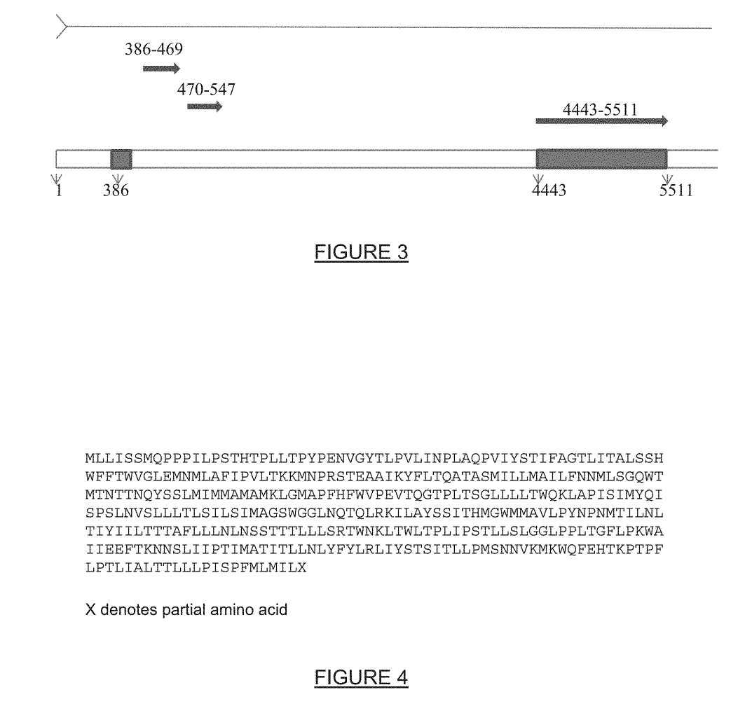

The 5′ contents of the Frame 2 fusion transcript starts with proline at position 386 but only initiates at the first methionine located at position 470 located within the Hypervariable segment 3 (438-574). The 3′ content of the fusion transcript begins at position 4443 within tRNA methionine (4402-4469) and terminates at position 5511 of NADH dehydrogenase subunit 2 (4470-5511) (FIG. 3). FIG. 4 displays the resulting fusion transcript initiating at position 470 formed by the deletion of positions 548-4442 of the mitochondrial genome.

Accordingly, the present invention encompasses fusions transcripts of varied content, i.e. those comprising spliced sequences of two coding regions and those comprising spliced coding and non-coding regions.

Fusion Transcripts Associated with UVR

Once mitochondrial fusion transcripts have been identified, the sequences are then tested for their association with UVR, as described in the examples below. Such testing may be carried out in vivo, or in cultured skin equivalents that are grown and dosed with varying levels of UVR (see Example 6). Expression of these sequences may also be monitored in respect of one or more house keeper transcripts including, but not limited to, Human PPIB, Human PGK-1, Human ACTB (beta actin), Human GUSB (Beta Glucuronidase), Human B2M (Beta-2-microglobulin), Human PPIA (peptidylprolyl isomerase A (cyclophilin A)), Human GAPD (glyeraldehyde-3-phosphate dehydrogenase), Human HPRT1 (hypoxanthine phosporibosyltransferase 1), Human RPLPO=LRP (ribosmal protein, large, PO), Human HMBS=PBGD (hydroxymethlbilane synthase), Human TBP (TATA box binding protein), Human TRFC (transferrin receptor (p90, CD71), and Human ABCC13 (Chromosome 13—psuedogene).

In one embodiment of the invention, the following mitochondrial fusion transcripts have been determined useful for predicting, diagnosing or monitoring UV damage, and for testing and screening skin care products effective in preventing or ameliorating UV damage:
SEQ ID NO: 22 (Transcript 2; 10744:14124)
SEQ ID NO: 23 (Transcript 3; 7974:15496)
SEQ ID NO: 31 (Transcript 11; 7775:13532)
SEQ ID NO: 32 (Transcript 12; 8213:13991)
SEQ ID NO: 38 (Transcript 20; 8469:13447; OrigMet)
SEQ ID NO: 39 (Transcript 32; 547:4443)

In addition to the above sequences, the present invention provides the use of variants or fragments of these sequences for predicting, diagnosing and/or monitoring UV exposure. Variants or fragments of the fusion transcripts identified herein adhere to the size limitations and percent identities described above with respect to the genomic variants and fragments, or as determined suitable by a skilled technician.

In addition, putative protein sequences corresponding to transcripts 2, 3, 11, 12, 20 and 32 of the invention are listed below. These sequences, which encode hypothetical fusion proteins, are provided as a further embodiment of the present invention and may be considered useful in the methods of the present invention.
SEQ ID NO: 40 (Transcript 2)
SEQ ID NO: 41 (Transcript 3)
SEQ ID NO: 42 (Transcript 11)
SEQ ID NO: 43 (Transcript 12)
SEQ ID NO: 44 (Transcripts 20)
SEQ ID NO: 45 (Transcript 32)

Primers and Probes

Once a fusion transcript has been characterized, primers or probes can be developed to target the transcript in a biological sample. Such primers and probes may be prepared using any known method (as described above) or as set out in the examples provided in the Applicant's co-pending PCT application. A probe may, for example, be generated for the fusion transcript, and detection technologies, such as QuantiGene 2.0™ by Panomics™, used to detect the presence of the transcript in a sample. Primers and probes may be generated directly against exemplary fusion transcripts of the invention, or to a fragment or variant thereof. For instance, the sequences set forth in SEQ ID NOs: 22, 23, 31, 32, 38 and 39 can be used to design probes that will detect a nucleic acid sequence comprising a fusion sequence of interest.

As would be understood by those skilled in the art, probes designed to hybridize to the fusion transcripts of the invention contain a sequence complementary to at least a portion of the transcript and preferably expressing the junction point of the spliced genes. This portion includes at least one of the nucleotides complementary to the expressed junction point, and may further comprise one or more complementary nucleotides adjacent thereto. In this regard, the present invention encompasses any suitable targeting mechanism that will select a fusion transcript that uses the nucleotides involved and adjacent to the junction point of the spliced genes.

Various types of probes and methods of labelling known in the art are contemplated for the preparation of transcript probes. Such types and methods have been described above with respect to the detection of genomic sequences. The transcript probes of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A probe of “at least 20 nt in length,” for example, is intended to include 20 or more contiguous bases that are complementary to an mtDNA sequence of the invention. Of course, larger probes (e.g., 50, 150, 500, 600, 2000 nucleotides) may be preferable.

The probes of the invention will also hybridize to the fusion transcripts in biological samples, thereby enabling the methods of the invention. Accordingly, in one aspect of the invention, there is provided a hybridization probe for use in detecting, diagnosing and/or monitoring UV damage or exposure, wherein the probe is complementary to at least a portion of a fusion transcript of the invention. In another aspect, there is provided probes and use thereof (or a method of using) such probes for testing and screening skin care products effective in preventing or ameliorating UV damage or exposure.

Assays

In accordance with the present invention, novel fusion transcripts have been demonstrated to increase in abundance following exposure to irradiation with solar simulated light (see, for instance, Example 6). Detection of these novel fusion transcripts both in vitro and in vivo allows for the quantification of exposure to UV irradiation (both UVB and UVA). The present invention, therefore, encompasses methods for detecting, diagnosing or monitoring UV damage or exposure, comprising obtaining one or more biological samples, extracting mitochondrial RNA from the samples, and assaying the samples for fusion transcripts by: quantifying the amount of one or more fusion transcripts in the sample and comparing the quantity detected with a reference value.

As would be understood by those of skill in the art, the reference value is based on whether the method seeks to detect, diagnosis or monitor UV damage or exposure. Accordingly, the reference value may relate to transcript data collected from one or more known UV exposed biological samples, from one or more known non-UV exposed biological samples, and/or from one or more biological samples taken over time. The sample may be derived from rarely sun exposed, occasionally sun exposed, usually sun exposed skin or blood and collected by a variety of methods as indicated above.

In one aspect, the invention provides a method of detecting UV exposure in a mammal, the method comprising assaying a tissue sample from the mammal for the presence of a fusion transcript described above. The present invention also provides for methods comprising assaying a tissue sample from the mammal by hybridizing the sample with at least one hybridization probe. The probe may be generated against a fusion transcript of the invention as described herein.

In another aspect, the invention provides a method as above, wherein the assay comprises: a) conducting a hybridization reaction using at least one of the probes to allow the at least one probe to hybridize to a complementary fusion transcript sequence; b) quantifying the amount of the at least one fusion transcript sequence in the sample by quantifying the amount of the transcript hybridized to the at least one probe; and, c) comparing the amount of the transcript in the sample to at least one known reference value.

As discussed above, the diagnostic assays of the invention may also comprise diagnostic methods and screening tools as described herein and can be readily adapted for high-throughput. The present invention, therefore, contemplates the use of the fusion transcripts and associated probes of the present invention in high-throughput screening or assays to detect and/or quantitate target nucleotide sequences in a plurality of test samples.

Testing and Screening of Skin Care Products

New Formulations

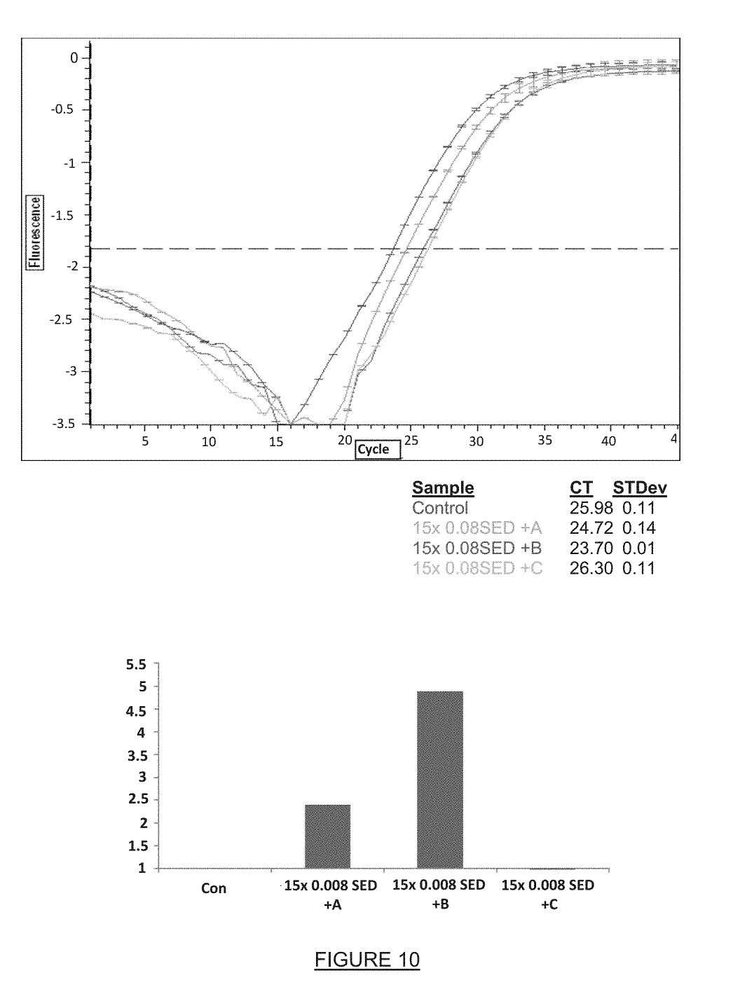

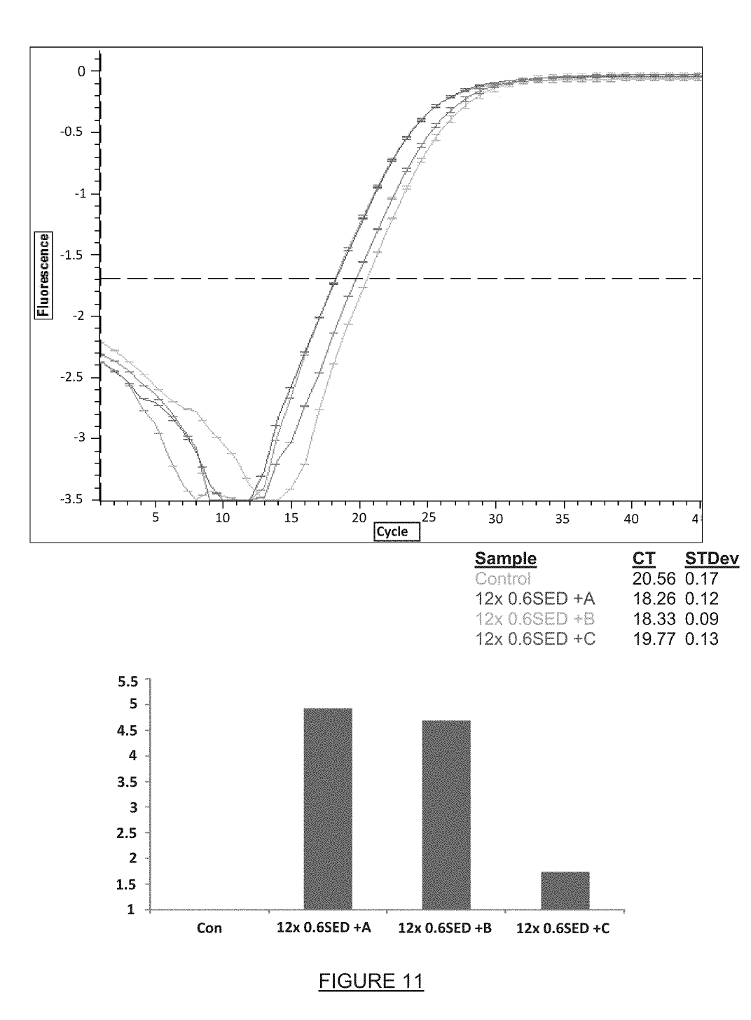

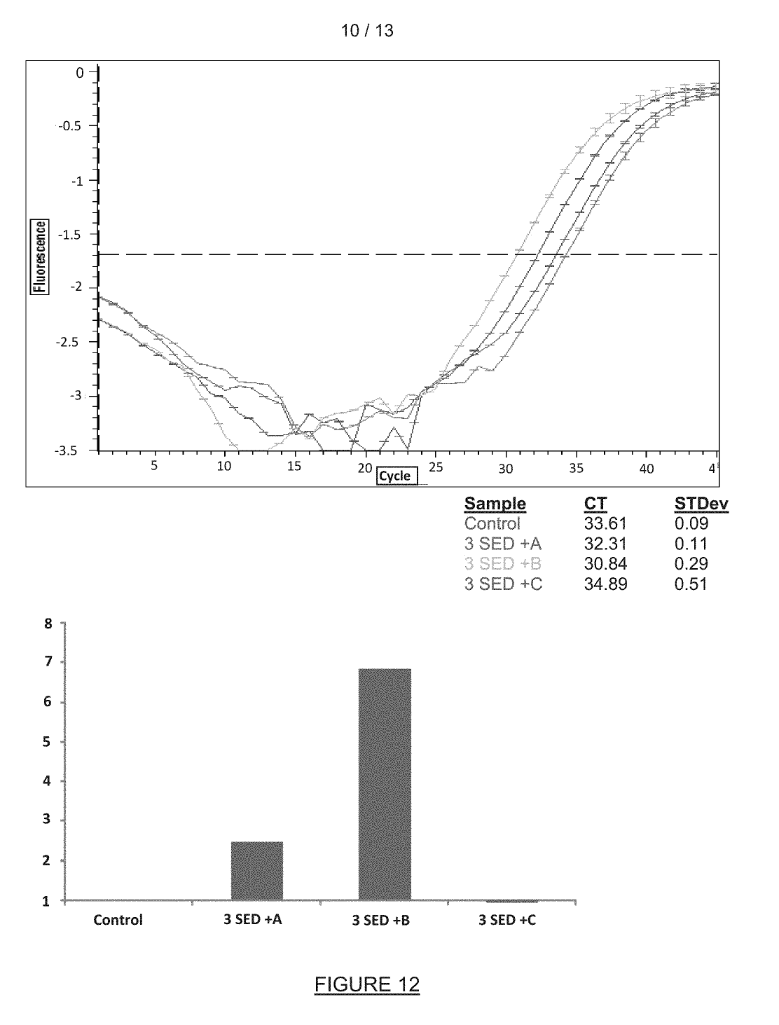

Modulation of changes in the abundance of novel UV associated fusion transcripts and related mtDNA molecules, through the application of specific actives or formulations such as sunscreens, anti-oxidant or anti-ageing products, may be used to assess the efficacy of products in protecting the skin from damage by UV irradiation. Such assessment may take the form of in vitro testing of new formulations using Skin Equivalent Models (see FIGS. 10 and 11) or through in vivo testing of products using biological samples (see FIG. 12).

The invention therefore contemplates methods of preparing and testing the efficacy of a skin care product to prevent, minimize, ameliorate or protect against UV exposure or damage by preparing a product having the desired characteristics (e.g. UVA filter and/or UVB filter), applying the skin care product to a patient's skin or to a skin equivalent model; exposing the skin or skin equivalent model to UVR; detecting the presence of at least one mitochondrial deletion molecule and/or fusion transcript in a sample taken from the exposed patient's skin or the exposed skin equivalent; and comparing the presence of the deletion or transcript to a reference value, such as a control gene and/or transcript. Control patients or skin equivalents models, which have not received the skin product, may also be used as a reference value.

The skilled person would understand that the arrest, prevention, decrease, or improvement in one or more the symptoms, signs, or features of UV damage, both temporary and long-term, indicates the ability of the new product (formulation) to prevent, minimize, ameliorate or protect against UV exposure or damage. In one embodiment of the invention, product analysis is performed as shown in the examples provided below.

In preferred embodiments of the invention, the methods for testing the new products comprise exposing a subject's skin or a skin equivalent model to at least one sub-lethal dose of ultraviolet radiation (UVR) prior to the step of detecting the presence of the mtDNA deletion or fusion transcript. Further, the skin or skin equivalent may be exposed to a series of repetitive sub-lethal doses of UVR, such as daily doses of UVR, prior to testing. Generally, the UVR is from a solar-simulated UVR source, wherein the UVR comprises UVA, UVB, or UVA/UVB, however, sun exposure is also herein contemplated. Also contemplated are control sequences including, but not limited to, the housekeeping genes and transcripts discussed above. As for the mtDNA deletion and fusion transcript markers, these can be inserted at the end of the described methods either singularly or in tandem.

Skin Samples

The patient's skin sample may be collected from the dermal or epidermal layer of the skin and may be derived by way of punch biopsy, surgical excision, and non-invasive or minimally invasive skin sampling methods such as a wet swabbing, tapelift, cotton tip swabbing, scraping of skin using a sterile surgical blade, scraping of skin using a wooden scraper, sticky surface of an adhesive pad (CapSure™ Clean-up Pad, Arcturus), film from LCM MacroCap™ (Arcturus), heated film from LCM MacroCap™ (Arcturus) and employing a small gauge needle (for example, 28 gauge), to collect micro-cores of skin tissue. The sample can be used either directly as obtained from the source or following a pre-treatment to modify the character of the sample. Thus, the skin sample can be pre-treated prior to use, for example, with preservatives, reagents, and the like.

One skilled in the art will appreciate that more than one sampling technique may be employed at a single time. Furthermore, where a course of collections are required, for example, for the monitoring of a product over time, the same or different techniques may be used alone or together throughout the test period. In this regard, skin collections may be taken once only, or at regular intervals such as daily, weekly or monthly.

Skin Equivalent Models

The Applicant has also developed a novel system for testing skin care products in vitro using Skin Equivalent Models. These models are produced and grown, for instance, as described in Example 2, following which they may be treated with a skin care product and dosed by varying increments of UVR. The product to be tested is simply applied to the surface of the skin equivalent, for example, at a density of 2 mg/cm², and spread evenly across the surface of the cells. The skin equivalents are then placed under the solar simulator and exposed to one or more doses of UV light. Dosing is dependent on the individual experimental setup and product to be tested. However, several examples of typical dosing regimes are provided in Tables 1 and 2 below.

Product Screening and Individualized Skin Care

Through the collection of data on the ability of skin care products to protect against the generation of fusion transcripts and related mtDNA deletions, more effective products with regard to sun protection and anti-ageing can be developed. As well, until more recently, sunscreens and sunblocks regularly applied by individuals during sun exposure generally protected against the mutagenizing effects of UVB, but failed to contain agents directed at the harmful effects of UVA radiation. Thus, targeted screening of skin care products will aid in identifying those capable of protecting against both UVA and UVB radiation.

These factors among others necessitate the availability of a tool to determine both the efficacy of new products entering the market and, through a course of studies, monitor the success and appropriateness of current measures to prevent UVR damage to the skin. Measuring the level of mitochondrial DNA deletions in the skin of a patient or skin equivalent model by collecting skin samples following product application and UVR dosing at regular intervals can assist with determining the efficacy of such products for the prevention of DNA and skin damage, including associated photoaging and skin cancer.

The methods of the present invention may also be used for widespread skin screening for both medical and cosmeceutical purposes. For example, the ability to assess the level of DNA damage in a subject's skin due to UV radiation at any time point and from any external anatomical location provides the foundation for a unique and informative screening test to assess the safety and efficacy of existing and new skin care products and skin care regimes for a given subject. Furthermore, by identifying the specific genetic changes associated with UV exposure, it may be readily determined whether and to what extent a particular skin care product or regime should be applied.

For skin care products already on the market, the methods of the present invention also assist in screening agents to gauge their ability to prevent, minimize, ameliorate or protect against UV exposure or damage. This allows a practitioner or consumer to assess which brands are best suited for their particular skin care requirements. Products to be screened by the methods of the invention include but are not limited to sunscreens and anti-aging serums and creams and maybe assessed in pairs or in batches of 3 or more products. Screening methods may be carried out as described above for testing new formulations or as detailed in the examples below.

Kits

The present invention provides diagnostic kits for detecting or monitoring the UV exposure of a subject. Such kits may include one or more sampling means, in combination with one or more primers or probes according to the present invention. Such kits may also include instructions for using the contents thereof.

The kits can optionally include reagents required to conduct a diagnostic assay, such as buffers, salts, detection reagents, and the like. Other components, such as solutions for the isolation and/or treatment of a biological sample, may also be included in the kit. One or more of the components of the kit may be lyophilised and the kit may further comprise reagents suitable for the reconstitution of the lyophilised components.

Where appropriate, the kit may also contain reaction vessels, mixing vessels and other components that facilitate the preparation of the test sample. The kit may also optionally include instructions for use, which may be provided in paper form or in computer-readable form, such as a disc, CD, DVD or the like.

In one embodiment of the invention there is provided a kit for diagnosing UV exposure comprising sampling means and a probe or primer of the invention.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Growth of HpEKp Cells

Materials
1. HpEKp Cells <15 passage (CellnTec)
2. T75 Tissue Culture Flasks (IWAKI)
3. TrypLE™ Select (12563-011, Invitrogen)
4. 10 ml Sterile Stripettes (Star Labs)
5. Automated Pipetter
6. Inverted TC microscope
7. Sterile 15 ml Falcon Tube (Falcon)
8. Class 2 Tissue Culture Cabinet [37° C., 5% CO²] (Binder)
9. Water Bath (37° C.)
10. Sterile Phosphate buffered Saline
11. Complete PCM Medium [CnT-57] (CellnTec)

Protocol
1. Remove CnT-57 medium from flasks of HpEKp cells (90% confluence)
2. Wash cells with sterile PBS (2 ml), aspirate off.
3. Add 1.5 ml TrypLE™ Select per flask, to cover cells.
4. Place back in incubator for 2-3 minutes, until cells become detatched (check with microscope).
5. Resuspend Cells in 5 ml of CnT-57 Medium to resuspend cells, vigorously pipette 2/3 times.
6. Spin the cells at 160×g for 5 min.
7. Remove medium and resuspend in 5 ml of fresh CnT-57 Medium, vigorously pipette 2/3 times.
8. Remove 20 ul cell suspension for cell counting.
9. Dilute cells and seed to a concentration of 4×10³ cells/cm²
10. Place Flasks in a humidified incubator at 37° C. and 5% CO2
11. Maintain cells by feeding with 15 ml CnT-57 medium every 2/3 days.
12. Grow until 90% confluent then passage again.

### Example 2: Epidermal Skin Equivalent Production

Materials
1. HpEKp Cells <15 passage (CellnTec) grown to 90% confluence (see Example 1)
2. T75 Tissue Culture Flasks (IWAKI)
3. TrypLE™ Select (12563-011, Invitrogen)
4. 10 ml Sterile Stripettes (Star Labs)
5. Automated Pipetter
6. Inverted TC microscope
7. Sterile 15 ml Falcon Tube (Falcon)
8. 6 Well Culture Plate (Millipore)
9. Millicell PCF 0.4 μm Inserts (Millipore)
10. RapiDiff II stain Pack (BioStain)
11. 24 Well Culture Plates (Millipore)
12. Class 2 Tissue Culture Cabinet [37° C., 5% CO2] (Binder)
13. Water Bath (37° C.)
14. Sterile Phosphate buffered Saline
15. Sterile Forceps
16. Complete PCM Medium [CnT-57] (CellnTec)
17. Complete 3D-Prime Medium [CnT-02-3DP] (CellnTec)

Protocol
1. Place four Millicell PCF 0.4 μm 12 mm inserts (Millipore Cat#: PIHP01250) into each well of a 6 well culture plate, plating out the number required for the experiment. Allow for two spare inserts (to monitor confluency—step 15).
2. Remove CnT-57 medium from flasks of HpEKp cells.
3. Wash cells with sterile PBS (2 ml), aspirate off.
4. Add 1.5 ml TrypLE™ Select per flask, to cover cells.
5. Place back in incubator for 2-3 minutes, until cells become detached (check with microscope).
6. Re-suspend Cells in 5 ml of CnT-57 Medium to re-suspend cells, vigorously pipette 2/3 times.
7. Spin the cells at 160×g for 5 min.
8. Remove medium and re-suspend in 5 ml of fresh CnT-57 Medium, vigorously pipette 2/3 times.
9. Remove 20 ul cell suspension for cell counting.
10. Dilute cells to a concentration of 5×10₅ cells per ml.
11. Add 2×105 cells in 400μl CnT-57 (or CnT-07) per insert.
12. Add the appropriate amount (˜2 ml) of CnT-57 outside the inserts (into the plate well), so that medium levels inside and outside the insert are equal and submerge the cells; make sure that no air bubbles are trapped underneath the membrane.
13. Place the inserts in a humidified incubator at 37° C. and 5% CO2
14. Allow the cells to grow for 2-3 days.
15. Stain Single spare insert with RAPI-DIFF II stain.
16. If the monolayer is confluent, proceed with step 17, otherwise change medium in the remaining inserts, cultivate for another day and perform then another staining with the second spare insert.
17. Replace the culture medium with 3D medium (CnT-02-3DP) inside and outside the insert (same amounts as when seeding, Step 12).
18. Place the inserts in the incubator overnight (15-16 h) to allow cells to form intercellular adhesion structures.
19. Initiate 3D cultures by aspirating all the medium from inside the insert and place into individual wells of a 24 well plate. Add (CnT-02-3DP) outside medium (250 μl)
20. If a time course study is performed, inserts can be left in the same plate with medium changes every 2-3 days. Grow cells for 14 days air dry prior to beginning dose treatments.

### Example 3: Epidermal Skin Equivalent Dosing—UVA Irradiation

Materials
1. Oriel 1000 W UV Solar Simulator (Newport Corp.)
2. Atmospheric Attenuation Filter (Newport Corp.—81017)
3. Vis IR Filter (Newport corp—87066)
4. PETG Filter (RVI Medical Physics)
5. International Light UVA Phototherapy Radiometer (Able Instruments—IL1402)
6. Epidermail Skin equivalent (See Example 2)
7. Complete 3D-Prime Medium [CnT-02-3DP] (CellnTec)
8. 24 Well Culture plate (Millipore)
9. Sterile Phosphate buffered Saline
10. Sterile Forceps and Scalpel

UV Spectrum Used (Solar Simulated)

Atmospheric Attenuation filter+Vis IR filter+PETG Filter: As shown in FIG. 15.

Procedure
1. Skin Equivalents produced and grown as set out in Example 2.
2. After 14 days of air dry growth the SEs are ready for dosing/treatment.
3. Skin equivalents are removed from the growth medium and placed in new 24 well plate, containing 200 μl of sterile PBS.
4. If required the product to be tested is applied to the surface of the skin equivalent, at a density of 2 mg/cm² (or at any specified dose). The material is applied with a bent sterile 200 μl pipette tip, and spread evenly across the surface.
5. Once the skin equivalent is prepared, they are placed back in the incubator for 20 minutes prior to exposure to UV light.
6. The solar simulator is turned on and allowed to warm up for 10 minutes prior to treatment. The UV output is measured with the radiometer and the intensity is used to calculate the required time of exposure.
7. The skin equivalents are placed under the solar simulator and exposed to X SED of UV light (as calculated in 6 dependant on experimental requirements).
8. Following exposure the skin equivalents are replaced into the 3D medium and back into the incubator.
9. Dosing is dependent on the individual experimental setup. Some examples are shown in table 1.
10. Following dosing SEs are removed from the 3D medium and stored at −80° C. prior to extraction.

TABLE 1
Experimental Setup

Dose Response 0.04 SED, multiple doses
Dose Response 0.08 SED, multiple doses
SPF 15 Products 0.08 SED, multiple doses
Anti-Aging products 0.08 SED, multiple doses

### Example 4: Epidermal Skin Equivalent Dosing—Solar Simulated Light

Materials
1. Oriel 1000 W UV Solar Simulator (Newport Corp.)
2. Atmospheric Attenuation Filter (Newport Corp.—81017)
3. Vis IR Filter (Newport corp—87066)
4. International Light UVA Phototherapy Radiometer (Able Instruments—IL1402)
5. Epidermail Skin equivalent (See Example 2)
6. Complete 3D-Prime Medium [CnT-02-3DP] (CellnTec)
7. 24 Well Culture plate (Millipore)
8. Sterile Phosphate buffered Saline
9. Sterile Forceps and Scalpel

UV Spectrum Used (Solar Simulated)

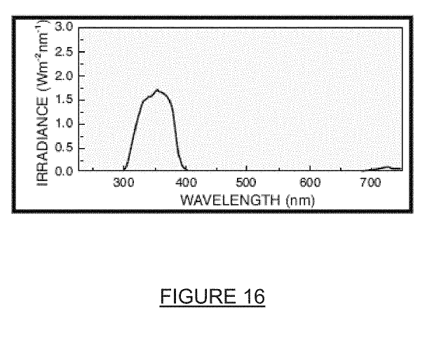

Atmospheric Attenuation filter+Vis IR filter: as shown in FIG. 16.

Procedure
1. Skin Equivalents produced and grown as set out in Example 2.
2. After 14 days of air dry growth the SEs are ready for dosing/treatment.
3. Skin equivalents are removed from the growth medium and placed in new 24 well plate, containing 200 μl of sterile PBS.
4. If required the product to be tested is applied to the surface of the skin equivalent, at a density of 2 mg/cm² (or at any specified dose). The material is applied with a bent sterile 200 μl pipette tip, and spread evenly across the surface.
5. Once the skin equivalent is prepared, they are placed back in the incubator for 20 minutes prior to exposure to UV light.
6. The solar simulator is turned on and allowed to warm up for 10 minutes prior to treatment. The UV output is measured with the radiometer and the intensity is used to calculate the required time of exposure.
7. The skin equivalents are placed under the solar simulator and exposed to X SED of UV light (as calculated in 6 dependant on experimental requirements).
8. Following exposure the skin equivalents are replaced into the 3D medium and back into the incubator.
9. Dosing is dependent on the individual experimental setup. Some examples are shown in table 1.
10. Following dosing SEs are removed from the 3D medium and stored at −80° C. prior to extraction.

TABLE 2
Experimental Setup

Dose Response 0.5 SED, multiple doses
Dose Response 1 SED, multiple doses
SPF 15 Products 1 SED, multiple doses
Anti-Aging products 0.5 SED, multiple doses

### Example 5: In Vivo UVR Dose Response

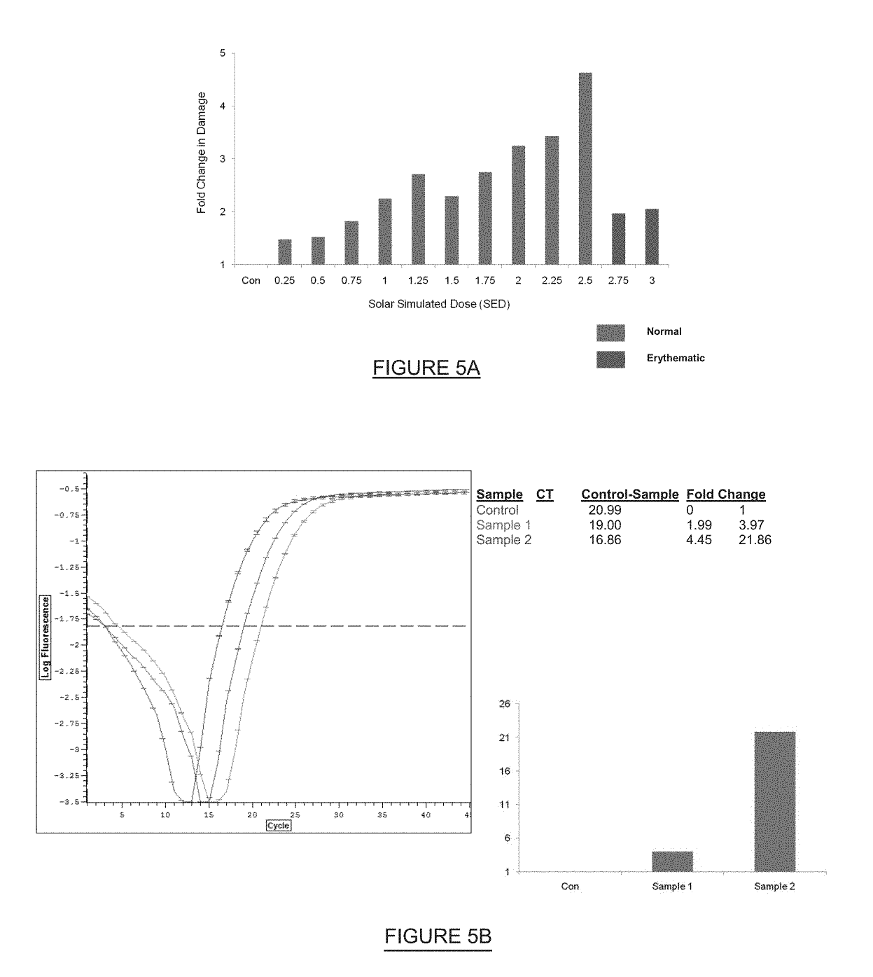

In vivo testing was carried out on individual(s) by dosing with various levels of UVR followed by swabbing to collect skin samples. Referring to FIG. 5, SED refers to Standard Erythemal Dose, which is the amount of UVR required to cause erythema (or reddening of the skin). FIG. 5A shows the dose response results for mtDNA deletion analysis performed by quantative realtime PCR on skin swab samples taken following UVR doses of up to 3.0 SED. This experiment demonstrates that UVR induced mtDNA damage increases with increasing doses of UVR, up to the point where erythema begins to occur. At this point the mtDNA damage in the sample falls, possibly due to increased levels of apoptosis in the erythemic tissue. In terms of long term damage to skin this shows that it is sub erythemal doses which create the long term mtDNA damage observed in skin. The fold change in damage is calculated as set out in FIG. 5B.

### Example 6: Identification of Fusion Transcripts Associated with UV Exposure

Cultured skin equivalents were grown and dosed with varying levels of UVR using a solar simulator as described in the examples above.

Samples were processed according to the manufacturer's protocol QuantiGene Sample Processing Kit: Cultured Cells (Panomics QS0100) and the QuantiGene 2.0 Reagent System (Panomics QS0008), and specifically 300 ul of dilute lysis mixture was added to each skin equivalent which were then incubated at 50 degrees Celsius for 1.5 hours or until the cells appeared to have completely lysed off of the membrane. Each was then diluted 1:10 and the remainder of the protocol carried out, targeting fusion transcripts 2, 3, 11, 12, 20 and 32 with ACTB Beta-Actin as the Housekeeper. Each sample was assayed in triplicate for all transcripts and a no-template background was established for each sample/transcript pair in triplicate as well.

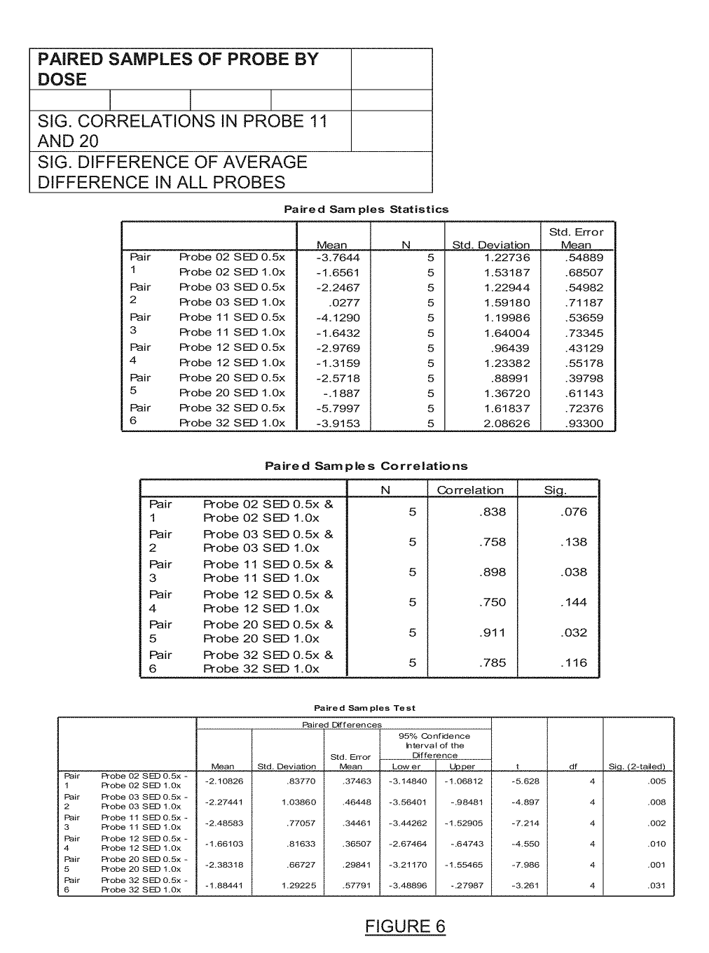

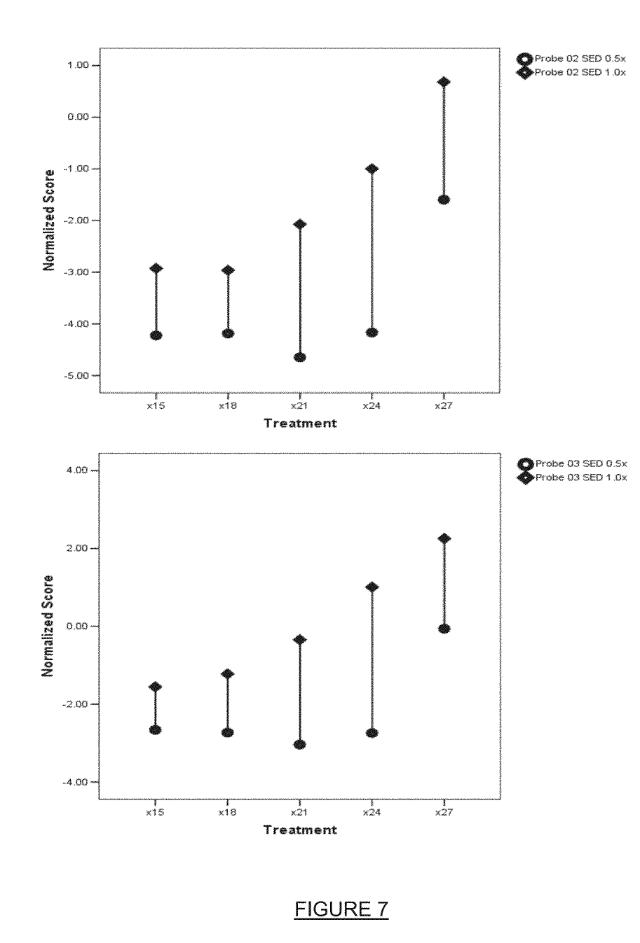

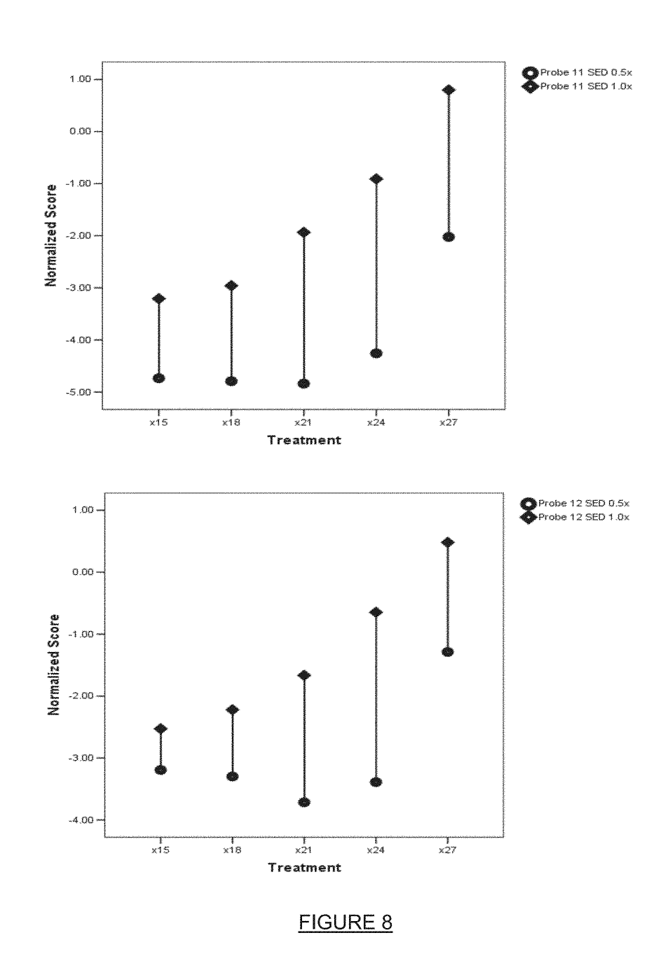

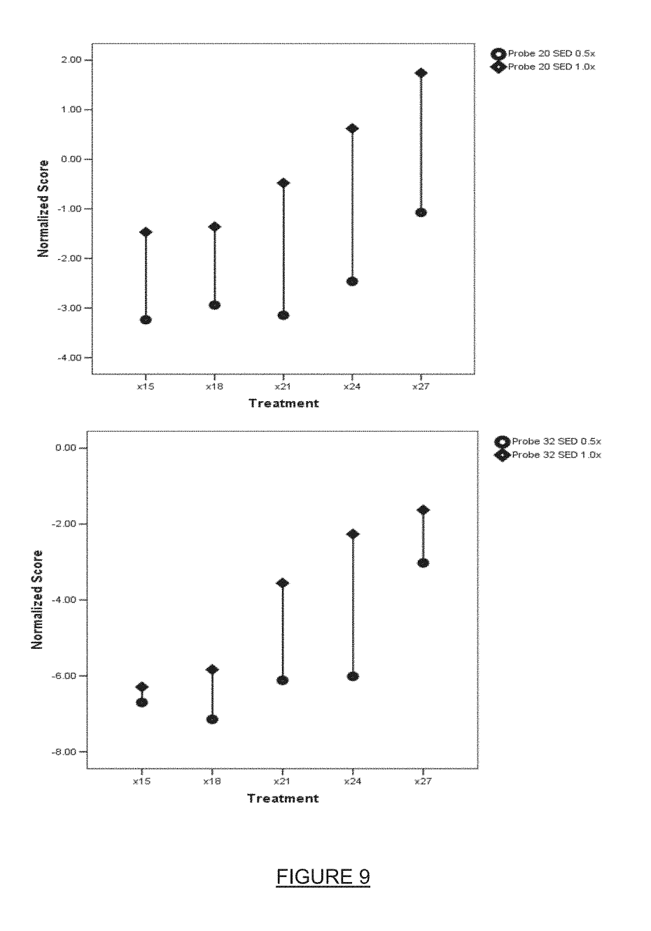

Referring to FIGS. 6-9, 0.5 SED and 1.0 SED were used for the purposes of this experiment at a various number of repeated doses (0×, 15×, 18×, 21×, 24×, 27×). For clarity, those skin equivalents exposed to 1.0 SED 27× received the greatest level of UVR.

These results demonstrate that each of the fusion transcripts tested increased in quantity both with increasing repeat doses as well as between the 0.5 and 1.0 SED indicating an association with UVR exposure.

### Example 7: Product Formulation and Testing

Three blinded formulations of varying levels of UVA filter were tested at 2 mg/cm2. Samples of each formulation were tested on 14 day old Skin Equivalents with both UVA and Solar Simulated light sources. As well, the samples were tested in vivo. Deletion analysis was performed by sybr green quantitative realtime PCR.

The experimental formulations were prepared in a cosmetic lotion emulsion comprised of standard cosmetic ingredients. Each product contains a constant level of UVB filter but levels of UVA filter were varied as follows:

TABLE 3
Content of new formulations

| Sample No. | UVB Filter | UVA Filter | Star Rating |
|---|---|---|---|
| 1 (B in figures) | 3.8% | 0% | 1 Star |
| 2 (A in figures) | 3.8% | 1.75% | 3 Star |
| 3 (C in figures) | 3.8% | 4.5% | 5 Star |

FIG. 10 shows the results of PCR analysis on in vitro samples exposed to UVA (FIG. 10) and solar simulated light (FIG. 11) following application of the formulations (A, B or C). In FIG. 12, the results following in vivo testing of the formulations are provided. These data show the multifactoral nature of UVR damage from solar simulated light, when isolated with only UVA exposure, damage levels follow the percentage of UVA protection afforded by the sunscreen. However when the exposure combines both UVA and UVB UVR in solar simulated light the damage ratios between samples changes. With respect to samples A and B, rather than A being higher than expected, B may be lower than expected as the lack of UVA filter combined with the UVB in the solar simulated light has taken the damage past the erythemic threshold (section 00121), meaning less damage is recorded in the sample.

These results also demonstrate that there exists an inverse correlation between the level of damage as indicated by the level of deletion and the amount of UVA filter in the compound. For clarity, less UVA filter, as in compound B, results in higher levels of the deletion.

### Example 8: Sunscreen and Anti-Aging Brand Screening

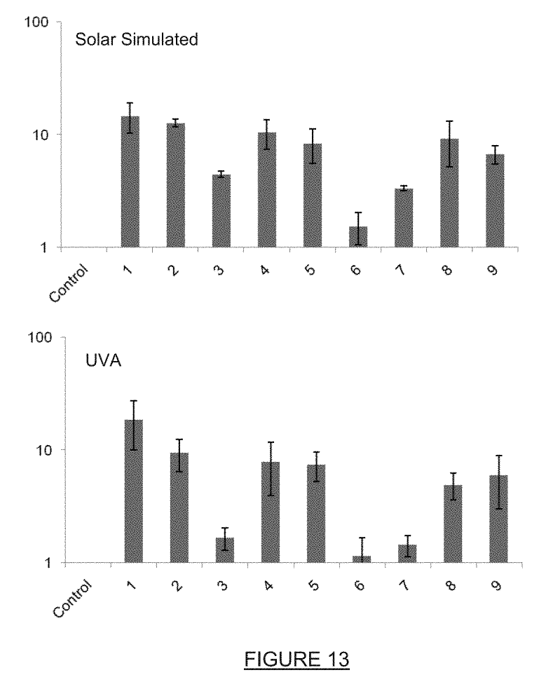

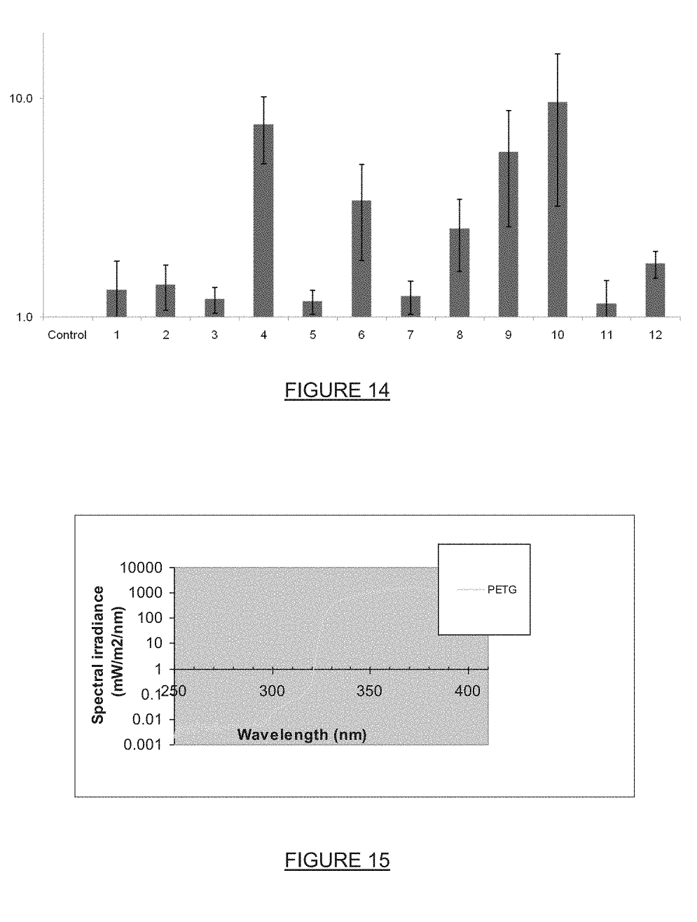

Samples of various sunscreen and anti-aging products were applied prior to repetitive exposure to UVA light at 2 mg/cm2. The panel of sunscreen agents included spf 15 products from top UK/North American brands. Anti-aging products included a range of night cremes/serums from a number of top brands (FIG. 13). The results show that despite all products having the same SPF rating (15), the level of protection afforded by the products varied considerably, following dosing with both UVA and solar simulated UVR. This suggests that SPF alone is insufficient to assess the effectiveness of UVR protection afforded by sunscreens, as other factors other than UVB exposure (measured by SPF rating) are important in mtDNA damage, such as UVA protection and anti-oxidant activity. Anti-aging products included a range of night cremes/serums from a number of top brands (FIG. 14). These data show that there is great variation in the protection afforded by anti-aging type products as far as mtDNA damage is concerned. Many of these products contain anti-oxidant compounds as their main actives, and this shows that measurement of mtDNA damage can distinguish between those formulations and indeed individual actives which are effective and those which exhibit little to no protection.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

### BIBLIOGRAPHY

The following references, amongst others, were cited in the foregoing description. The entire contents of these references are incorporated herein by way of reference thereto.

| Author | Journal | Title | Volume | Date |
|---|---|---|---|---|
| Anderson et al | Nature | Sequence and Organization of the Human | 290(5806): 457-65 | 1981 |
| | | Mitochondrial Genome | | |
| Andrews et al | Nat Genet | Reanalysis and revision of the Cambridge | 23(2): 147 | 1999 |
| | | reference sequence for human | | |
| | | mitochondrial DNA. | | |
| Modica- | Expert Rev | Mitochondria as targets for detection and | 4: 1-19 | 2002 |
| Napolitano et al | Mol Med | treatment of cancer | | |
| Sherratt et al | Clin Sci (Lond) | Mitochondrial DNA defects: a widening | 92(3): 225-35 | 1997 |
| | | clinical spectrum of disorders. | | |
| Croteau et al | Mutat Res | Mitochondrial DNA repair pathways. | 434(3): 137-48 | 1999 |
| Dai et al | Acta | Correlation of cochlear blood supply with | 24(2): 130-6 | 2004 |
| | Otolaryngol | mitochondrial DNA common deletion in | | |
| | | presbyacusis. | | |
| Ro et al | Muscle Nerve | Deleted 4977-bp mitochondrial DNA | 28(6): 737-43 | 2003 |
| | | mutation is associated with sporadic | | |
| | | amyotrophic lateral sclerosis: a hospital- | | |
| | | based case-control study. | | |
| Barron et al | Invest | Mitochondrial abnormalities in ageing | 42(12): 3016-22 | 2001 |
| | Ophthalmol | macular photoreceptors. | | |
| | Vis Sci | | | |
| Lewis et al | J Pathol | Detection of damage to the mitochondrial | 191(3): 274-81 | 2000 |
| | | genome in the oncocytic cells of Warthin's | | |
| | | tumour. | | |
| Muller-Hocker | Mod Pathol | The common 4977 base pair deletion of | 11(3): 295-301. | 1998 |
| et al | | mitochondrial DNA preferentially | | |
| | | accumulates in the cardiac conduction | | |
| | | system of patients with Kearns-Sayre | | |
| | | syndrome. | | |
| Porteous et al | Eur J Biochem | Bioenergetic consequences of accumulating | 257(1): 192-201 | 1998 |
| | | the common 4977-bp mitochondrial DNA | | |
| | | deletion. | | |
| Birch-Machin | Online | International Congress of Biochemistry and | | 2000(a) |
| M A | Conference | Molecular Biology, New Scientist | | |
| | Report | | | |
| | (Sunburnt | | | |
| | DNA) | | | |
| Lee H C et al. | Federation of | Aging-and smoking-associated alteration in | 441: 292-296 | 1998 |
| | European | the relative content of mitochondrial DNA in | | |
| | Biochemical | human lung | | |
| | Societies | | | |
| Polyak Y. et al. | Nature | Somatic mutations of the mitochondrial | 20 (3): 291-293 | 1998 |
| | Genetics | genome in human colorectal tumours | | |
| Rees J L | The Genetic | Skin Cancer | pp. 527-536 | 1998 |
| | Basis of | | | |
| | Human | | | |
| | Cancer | | | |
| Weinstock M A | Epidemiology | Epidemiology of Ultraviolet Radiation | pp. 121-128 | 1998 |

We claim:

1. An isolated mitochondrial fusion transcript associated with UV exposure, wherein the transcript has the nucleic acid sequence set forth in SEQ ID NO: 22, SEQ ID NO: 23, SEQ ID NO: 31, SEQ ID NO: 32, SEQ ID NO: 38 or SEQ ID NO: 39.

2. The fusion transcript of claim 1, wherein the transcript has the nucleic acid sequence set forth in SEQ ID NO: 39.

3. A kit for detecting UV exposure, the kit comprising:
a probe having a nucleic acid sequence substantially complementary to at least a portion of the nucleic acid sequence of SEQ ID NO: 3, SEQ ID NO: 4, SEQ ID NO: 12, SEQ ID NO: 13, SEQ ID NO: 19, SEQ ID NO: 22, SEQ ID NO: 23, SEQ ID NO: 31, SEQ ID NO: 32, SEQ ID NO: 38 or SEQ ID NO: 39; and
a reagent for use with the probe.

4. The kit of claim 3, wherein the probe has a nucleic acid sequence substantially complementary to at least a portion of the nucleic acid sequence of SEQ ID NO: 39.

5. A method of detecting or monitoring ultraviolet radiation (UVR) exposure in a biological sample the method comprising detecting the presence of a mitochondrial fusion transcript in the biological sample, wherein the transcript is associated with UVR exposure and has a nucleic acid sequence as set forth in SEQ ID NO:39.

6. The method of claim 5, wherein the biological sample is a skin sample taken from a subject.

7. The method of claim 5, wherein the biological sample is a tissue culture sample.

8. The method of claim 6, wherein the skin sample is taken from an epidermis layer of the subject.

9. The method of claim 5 further comprising exposing the sample to at least one sub-lethal dose of ultraviolet radiation (UVR) prior to the step of detecting the presence of the fusion transcript.

10. The method of claim 9, wherein the biological sample is exposed to a series of repetitive sub-lethal doses of UVR.

11. The method of claim 10, wherein the series of repetitive sub-lethal doses comprises exposing the biological sample to daily doses of UVR.

12. The method of claim 9, wherein the UVR is from a solar-simulated UVR source.

13. The method of claim 9, wherein the UVR comprises UVA, UVB, or UVA/UVB.

14. A method for determining the cumulative UV exposure in a subject, the method comprising detecting the presence of a mitochondrial fusion transcript associated with UV exposure in biological samples obtained from the subject over a period of time, wherein the transcript is associated with UVR exposure and has a nucleic acid sequence as set forth in SEQ ID NO:39.

15. The method of claim 14, wherein the biological samples are from rarely sun exposed, occasionally sun exposed, or usually sun exposed skin.

16. A method of testing or screening the ability of a skin care product to prevent, minimize, ameliorate or protect against UV exposure, UV damage, skin aging or photo-aging, the method comprising the steps of:
(a) applying the skin care product to a test sample, the test sample comprising skin or a skin equivalent;
(b) exposing the test sample to ultraviolet radiation (UVR);
(c) detecting in the test sample the presence of a mitochondrial fusion transcript associated with UV exposure and having a nucleic acid sequence as set forth in SEQ ID NO:39; and,
(e) comparing the result of the detection step with a reference value.

17. The method of claim 16, wherein the UVR is from a solar-simulated UVR source.

18. The method of claim 16, wherein the UVR comprises UVA, UVB, or UVA/UVB.
